(19) United States
(12) Patent Application Publication
(10) Pub. No.: US 2024/0261061 A1
(43) Pub. Date: Aug. 8, 2024

(54) SYSTEMS AND METHODS FOR CLEANING LUMENS WITH FLUIDIC COMPOSITIONS

(71) Applicant: Saban Ventures Pty Limited, Macquarie Park, NSW (AU)

(72) Inventors: Amir Hossein KAYVANPOUR, Carlingford, NSW (AU); Mohsen NABIPOOR, Mount Colah, NSW (AU); Gavin SPARGO, Box Hill, NSW (AU); Taylor BARKER, Newtown, NSW (AU)

(21) Appl. No.: 18/567,202

(22) PCT Filed: Jun. 9, 2022
(86) PCT No.: PCT/AU2022/050568

§ 371 Date: Dec. 5, 2023

(30) Foreign Application Priority Data
Jun. 9, 2021 (AU) 2021901729

(51) Int. Cl. A61B 90/70; A61L 2/18

(52) U.S. Cl. CPC A61B 90/70; A61L 2/18; A61B 2090/701

(57) ABSTRACT

Presented herein are techniques for cleaning the interior/internal lumens (e.g., channels, cylinders, valve sockets and connectors, etc.) of an apparatus, such as a medical device (medical instrument), using contaminant-detaching fluidic compositions. In particular, the techniques presented herein introduce an apportioned amount of a contaminant-detaching fluidic composition into an interior lumen of a medical device. The contaminant-detaching fluidic composition is configured, and propelled through at least a portion of the interior lumen device, so that the contaminant-detaching fluidic composition can clean the walls of the interior lumen (e.g., interact with the walls of interior lumen to remove contaminants there from).

### CROSS-REFERENCE TO RELATED APPLICATIONS

This application claims priority to Australian Patent Application No. 2021901729, entitled “Systems and methods for cleaning a medical device having a lumen using abrasive fluidic compositions,” filed Jun. 9, 2021, and to Australian Patent Application No. 2021901734, entitled “Systems and methods for the identification, evaluation, and/or closed-loop cleaning of lumens,” filed Jun. 9, 2021. This application also incorporates by reference the content of the concurrently filed patent application entitled “Systems and Methods for the Identification, Evaluation, and/or Closed-Loop Reprocessing of Lumens” and the content of International Patent Application No. PCT/AU2022/050547 entitled “Medical Device Port Connectors,” filed on Jun. 3, 2022.

### BACKGROUND

### Field of the Invention

The present invention generally relates to techniques for cleaning interior lumens of, for example, medical devices such as endoscopes.

### Related Art

Any discussion of the prior art throughout the specification should in no way be considered as an admission that such prior art is widely known or forms part of common general knowledge in the field.

There are several different types of medical devices (medical instruments) that can be used to perform diagnostic and/or surgical procedures. For example, an endoscope is a medical device that can be used to visually inspect hollow organs or body cavities. Specially designed endoscopes are used for different examinations, such as bronchoscopy, cystoscopy, gastroscopy, and proctoscopy. Endoscopes, as well as other available diagnostic and/or surgical medical devices are re-useable across multiple patients and include one or more interior lumens that must be cleaned between uses.

### SUMMARY

According to a first aspect of the present invention, there is provided a method for cleaning at least one interior lumen of a medical device comprising:
mixing a liquid with a powder to form a slurry; and
applying at least one flow of fluid to a portion of the slurry to propel the portion of the slurry through the at least one interior lumen of the medical device.

According to a second aspect of the present invention, there is provided a method, comprising:
apportioning a liquid-powder mixture into a cleaning slug; and
delivering the cleaning slug to a proximal end of at least one lumen so that the cleaning slug passes from the proximal end to a distal end of the at least one lumen.

According to a third aspect of the present invention, there is provided a system, comprising:
a holding chamber configured to retain a liquid-powder mixture therein;
at least one delivery chamber fluidically connected to at least one interior lumen of an apparatus;
at least one of a valve or pump configured to provide an apportioned amount of the liquid-powder mixture to the at least one delivery chamber; and
a delivery mechanism configured to apply at least one flow of fluid to the apportioned amount of the liquid-powder mixture in the at least one delivery chamber to propel the apportioned amount of the liquid-powder mixture through the at least one interior lumen.

In one aspect, a method for cleaning at least one interior lumen of a medical device is provided. The method includes mixing a liquid with a powder to form a slurry; and applying at least one flow of fluid to a portion of the slurry to propel the portion of the slurry through the at least one interior lumen of the medical device.

In another aspect, a method is provided. The method includes apportioning a liquid-powder mixture into a cleaning slug; and delivering the cleaning slug to a proximal end of at least one lumen so that the cleaning slug passes from the proximal end to a distal end of the at least one lumen.

In another aspect, a system is provided. The system includes a holding chamber configured to retain a liquid-powder mixture therein; at least one delivery chamber fluidically connected to at least one interior lumen of an apparatus; at least one of a valve or pump configured to provide an apportioned amount of the liquid-powder mixture to the at least one delivery chamber; and a delivery mechanism configured to apply at least one flow of fluid to the apportioned amount of the liquid-powder mixture in the at least one delivery chamber to propel the apportioned amount of the liquid-powder mixture through the at least one interior lumen.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWING

Embodiments of the present invention are described herein in conjunction with the accompanying drawings, in which:

FIG. 1 is a schematic diagram illustrating an endoscope with interior lumens that can be cleaned using aspects of the techniques presented herein.

FIG. 2A is a flowchart of an example method for cleaning an interior lumen of a medical device using contaminant-detaching fluidic compositions, in accordance with certain embodiments of the present invention.

FIG. 2B is a schematic diagram illustrating a first stage/phase of a process for cleaning a lumen, in accordance with certain embodiments of the present invention.

FIG. 2C is a schematic diagram illustrating a second stage/phase of a process for cleaning a lumen, in accordance with certain embodiments of the present invention.

FIG. 3 is a flowchart of a method for cleaning an interior lumen of a medical device using a contaminant-detaching fluidic composition created in a chamber pre-filled with at least one constituent component, in accordance with certain embodiments of the present invention.

FIG. 4 illustrates a system for cleaning an interior lumen of a medical device using a contaminant-detaching fluidic composition where a delivery mechanism is used to propel a portion of the contaminant-detaching fluidic composition through a target lumen, in accordance with certain embodiments of the present invention.

FIG. 5 illustrates another system for cleaning an interior lumen of a medical device using a contaminant-detaching fluidic composition where a delivery mechanism in the form of a delivery chamber is used to propel a portion of the contaminant-detaching fluidic composition through a target lumen, in accordance with certain embodiments of the present invention.

FIG. 6A illustrates another system for cleaning an interior lumen of a medical device using a contaminant-detaching fluidic composition where the contaminant-detaching fluidic composition is created in a consumable chamber, in accordance with certain embodiments of the present invention.

FIG. 6B illustrates yet another system for cleaning an interior lumen of a medical device using a contaminant-detaching fluidic composition where the contaminant-detaching fluidic composition is created in a consumable chamber, in accordance with certain embodiments of the present invention.

FIG. 7 illustrates a consumable chamber for retaining a contaminant-detaching fluidic composition for use in cleaning an interior lumen of a medical device, in accordance with certain embodiments of the present invention.

FIG. 8 illustrates another system for cleaning an interior lumen of a medical device using contaminant-detaching fluidic compositions where a delivery manifold is used to deliver the contaminant-detaching fluidic composition to the target lumen(s) to be cleaned, in accordance with certain embodiments presented herein.

FIG. 9 illustrates yet another system for cleaning an interior lumen of a medical device using contaminant-detaching fluidic compositions where a delivery manifold is used to deliver the contaminant-detaching fluidic composition to the target lumen(s) to be cleaned, in accordance with certain embodiments presented herein.

FIG. 10 is a schematic diagram illustrating air and water channels of an example endoscope that can be cleaned using contaminant-detaching fluidic compositions, in accordance with certain embodiments presented herein.

FIG. 11 is a schematic diagram illustrating a fluid delivery connector and modulation of cleaning slugs with fluidically complex lumens, in accordance with certain embodiments presented herein.

FIG. 12 is a flowchart of an example method for cleaning a fluidically complex lumen using a contaminant-detaching fluidic composition, in accordance with certain embodiments of the present invention.

+

FIG. 13 is a flowchart of another example method for cleaning a fluidically complex lumen using a contaminant-detaching fluidic composition, in accordance with certain embodiments of the present invention.

FIG. 14 is a block diagram of an example control sub-system for use in cleaning an interior lumen of a medical device using contaminant-detaching fluidic compositions, in accordance with certain embodiments presented herein.

### DETAILED DESCRIPTION

Presented herein are techniques for cleaning the interior/internal lumens (e.g., channels, cylinders, valve sockets and connectors, etc.) of an apparatus, such as a medical device (medical instrument), using “contaminant-detaching fluidic composition,” sometimes referred to simply herein as “fluidic compositions.” As used herein, the contaminant-detaching fluidic compositions described herein generally include solid particles (e.g., powder).

In accordance with embodiments presented herein, one or more apportioned amounts of a contaminant-detaching fluidic composition are introduced into an interior lumen of a medical device. The apportioned amounts of the contaminant-detaching fluidic composition are configured, and propelled through at least a portion of the interior lumen device, so that the apportioned amounts clean the walls of the interior lumen. That is, the particles within the contaminant-detaching fluidic composition can physically detach contaminants from the lumen wall.

Merely for ease of illustration, the techniques presented herein are primarily described with reference to cleaning a specific type of lumen, namely the channels of an endoscope. However, it will be appreciated that the invention is not limited to use with endoscopes or, more generally, to only use with medical devices. As such, it is to be appreciated that the techniques presented herein can be used to clean lumens of a number of different devices/instruments used in any of a number of different applications.

An endoscope is an elongate tubular medical device that may be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to carry out diagnostic and surgical procedures. Endoscopic procedures are increasingly popular as they are minimally invasive in nature and provide a better patient outcome (through reduced healing time and exposure to infection) enabling hospitals and clinics to achieve higher patient turnover.

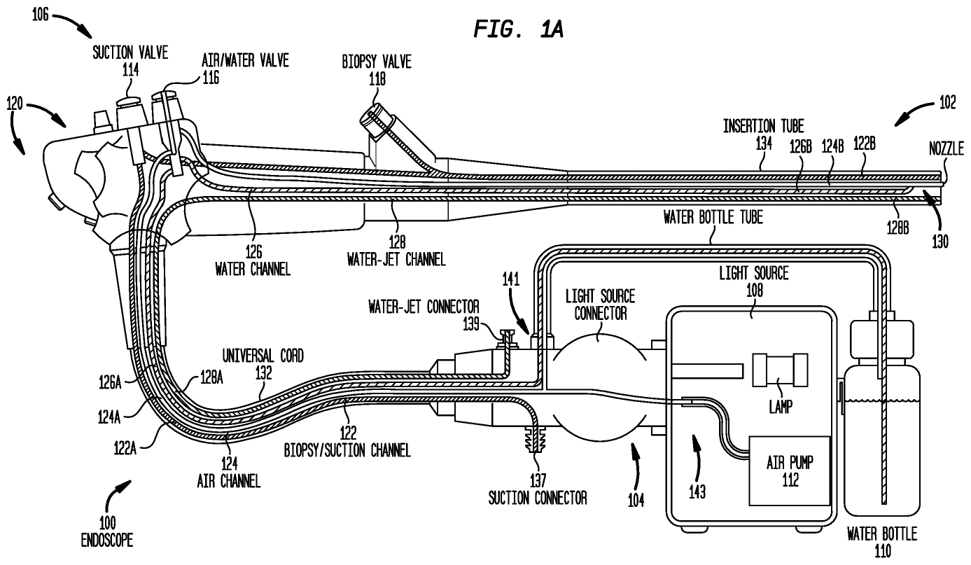

FIG. 1 is a schematic diagram of an example endoscope 100 with which aspects of the techniques presented herein can be implemented. As shown, endoscope 100, similar to most endoscopes, has a long tube-like structure with a distal end/tip 102 at one end for insertion into a patient and an opposing proximal or connector end 104, with a control handle 106 located between the two ends (e.g., generally at the center of the length between connector end 104 and distal end 102). The connector end 104 includes a plurality of connectors that enable the endoscope to be attached to, for example, a light source 108, water source 110, a suction source (not shown in FIG. 1), and a pressurized air source 112. For example, shown in FIG. 1A, is a suction port/connector 137, a water-jet (auxiliary) port/connector 139, a water port/connector 141, and an air port/connector 143. The control handle 106 is held by the operator during the procedure to control the endoscope 100 via valves, which include in this example a suction valve 114, an air/water valve 116, and a biopsy valve 118, and control wheels 120.

As shown in FIG. 1, endoscope 100 includes internal channels used either for delivering air and/or water, providing suction or allowing access for forceps and other medical equipment required during the procedure. As such, the distal tip 102 contains the camera lens (not shown in FIG. 1), and the exits for the lighting, air, and water, as well as exits for suction and forceps. Some of the internal channels run from one end of the endoscope 100 to the other, while others run via valve sockets at the control handle. Some channels bifurcate while and others join from two into one.

More specifically, shown in FIG. 1 is a biopsy/suction channel 122, an air channel 124, a water channel 126, and a water-jet channel 128. The biopsy/suction channel 122 includes two sections, referred to as proximal section 122A and distal section 122B that are connected via the suction valve 114. The air channel 124 also includes two sections, referred to as proximal section 124A and distal section 124B that are connected via the air/water valve 116. Similarly, the water channel 126 also includes two sections, referred to as proximal section 126A and distal section 126B that are connected via the air/water valve 116. The distal section 126B of the water channel joins to the distal section 124B of the air channel at a location 130 within the distal end 102. The water-jet channel 128 extends directly from the connector end 104 to the distal end 102 (via the control handle 106) but is similarly referred to as having a proximal section 128A and distal section 128B. The proximal sections 122A, 124A, 126A, and 128A of the channels are sometimes referred to as being located within a universal cord section (cord) 132 of the endoscope 100, while the distal sections 122B, 124B, 126B, and 128B of the channels are sometimes referred to as being located within an insertion tube 134 of the endoscope. More generally, as used herein, the proximal sections 122A, 124A, 126A, and 128A are the portions of the channels located between the connector end 104 and a valve (e.g., valve 114 or 116) at the control handle 106 and/or a mid-point of the control handle 106, as applicable. The distal sections 122B, 124B, 126B, and 128B are the portions of the channels located between the valve (e.g., valve 114 or 116) at the control handle 106 and/or a mid-point of the control handle 106, and the distal end 102 of the endoscope 102.

The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope 100, but also cleaning and disinfecting the internal channels/lumens (e.g., lumens 122, 124, 126, and 128 of FIG. 1).

Endoscopes used for colonoscopic procedures are typically between 2.5 and 4 meters long and have one or more lumen channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application, such as colonoscopes inserted into the colon, bronchoscopes inserted into the airways, gastroscopes for investigation of the stomach, etc. Gastroscopes, for instance, are smaller in diameter than colonoscopes; bronchoscopes are smaller again and shorter in length while duodenoscopes have a different tip design to access the bile duct.

A variety of options are available to mechanically remove biological residues from the lumen which is the first stage in the cleaning and disinfection process. By far the most common procedure for cleaning the lumens utilizes small brushes mounted on long, thin, flexible lines. Brushing is the mandated means of cleaning the lumen in some countries. These brushes are fed into the lumens while the endoscope is submerged in warm water and a cleaning solution. The brushes are then pushed/pulled through the length of the lumens in an effort to scrub off the soil/bio burden. Manual back and forth scrubbing is typically required. Water and cleaning solutions are then flushed down the lumens. These flush-brush processes are repeated three times or until the endoscope reprocessing technician is satisfied that the lumen is clean. At the end of this cleaning process air is pumped down the lumens to dry them. A flexible pull-through device having wiping blades may also be used to physically remove material. A liquid flow through the lumen at limited pressure can also be used.

In general, however, only the larger suction/biopsy lumens (e.g., 122 in FIG. 1) can be cleaned by brushing or pull throughs. Air/water channels (e.g., channels 124 and 126) are too small for brushes so these lumens are usually only flushed with water and cleaning solution.

After mechanical cleaning, a chemical clean is carried out to remove the remaining biological contaminants. Because endoscopes are sensitive and expensive medical instruments, the biological residues cannot be treated at high temperatures or with strong chemicals. For this reason, the mechanical cleaning needs to be as thorough as possible. In many cases, the current mechanical cleaning methodologies fail to fully remove biofilm from lumens, particularly where cleaning relies on liquid flow alone. Regardless of how good the conventional cleaning processes are, it is almost inevitable that a small microbial load will remain in the channel.

There has been significant research to show that the method of cleaning with brushes, even when performed as prescribed, does not completely remove biofilm in endoscope lumens. As well as lacking in efficacy, the current manual brushing procedures suffer from other drawbacks. The large number of different endoscope manufacturers and models results in many minor variations of the manual cleaning procedure. This has led to confusion and ultimately poor compliance in cleaning processes. The current system of brushing is also hazardous in that the chemicals that are currently used to clean endoscopes can adversely affect the reprocessing staff.

The current system of manual brushing is also labor intensive, leading to increased cost. Thus, the current approaches to cleaning and disinfecting the lumens in medical cleaning apparatus are still inadequate and residual microorganisms are now recognized as a significant threat to patients and staff exposed to these devices. For example, there is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked to the scopes, affecting 300 to 350 patients (http://www.modernhealthcare.com/article/20167415/NEWS/167419935). It would be expected that a reduction in the bioburden in various medical devices would produce a concomitant overall reduction in infection rates and mortality.

In addition, if endoscopes are not properly cleaned and dried, biofilm can build up on the lumen wall. Biofilms start to form when a free-floating microorganism attaches itself to a surface and surrounds itself with a protective polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes lumens are particularly prone to biofilm formation. They are exposed to significant amounts of bioburden, and subsequent cleaning of the long narrow lumens is quite difficult due to inaccessibility and the inability to monitor the cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

It is an object of the techniques presented herein to overcome or ameliorate at least one of the disadvantages of the prior art, or to provide a useful alternative. In particular, and as described in greater detail below, the techniques presented herein include systems and methods for cleaning a medical device, such as an endoscope, having interior lumens (e.g., channels, ports/cylinders, etc.). For example, certain embodiments relate to the use of contaminant-detaching fluidic compositions propelled through respective lumens of a medical device. It has been determined that certain contaminant-detaching compositions, and related techniques, are particularly effective at removing unwanted matter via physical contact—yet safely—interacting with the lumens to clean them. Thus, for example, techniques described herein may be used to remove biofilm that may be present within a lumen of a medical device.

Accordingly, in accordance with certain embodiments, a method of cleaning an interior lumen of a medical device is provided. This method comprises: creating a liquid-powder mixture (contaminant-detaching fluidic composition); apportioning the liquid-powder mixture into a suitable amount; and delivering the apportioned liquid-powder mixture at a suitable velocity through at least a portion of the lumen. As the cleaning efficacy/efficiency of a propelled apportioned amount of the liquid-powder mixture (e.g., portion of the contaminant-detaching fluidic composition) may be proportional to the velocity at which it flows through the lumen, it can be appreciated that a ‘suitable velocity’ or an ‘appropriate velocity’ refers to a relatively high velocity given the constraints of the fluidic characteristics of the lumen and the mechanical constraints of the lumen (e.g., the pressure ceiling).

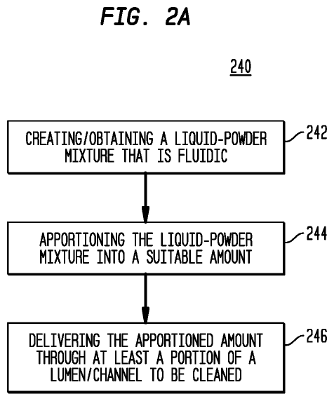

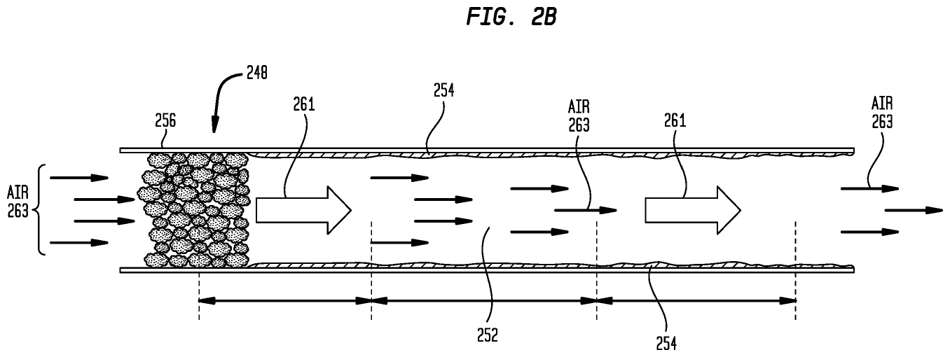

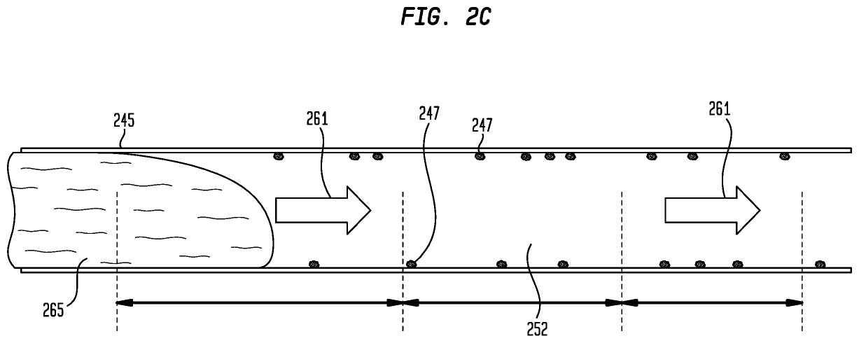

The liquid-powder mixture (e.g., contaminant-detaching fluidic composition) is sometimes referred to herein as a ‘slurry,’ and the apportioned amount of the liquid-powder mixture is sometimes referred herein to as a ‘cleaning slug’ or ‘slug.’ Thus, FIG. 2A illustrates an exemplary method 240 of cleaning a lumen of a medical device, in accordance with embodiments of the invention. It is to be appreciated that, as used herein, reference to ‘cleaning’ of a lumen refers to cleaning of the internal/interior portion of the lumen, including the inner surface or ‘walls’ forming/defining the lumen.

The method 240 of FIG. 2A begins at 242 with the creating, mixing, or otherwise obtaining of a liquid-powder mixture. At 244, the liquid-powder mixture is apportioned into a suitable amount. At 246, the apportioned amount of the liquid-powder mixture is delivered (e.g., propelled) through at least a portion of a lumen to be cleaned. This process may of course be implemented in any of a variety of ways in accordance with embodiments of the invention.

For example, any suitable liquid-powder mixture may be implemented. As can be appreciated, the liquid component of the mixture can facilitate the fluidity of the mixture, while the presence of the powder can act to interact with (e.g., scour) the walls of the target lumen (e.g., channel) to thereby clean the lumen. In accordance with various embodiments presented herein, the powder component of the liquid-powder mixture is present within the mixture in amounts greater than the respective saturation limit within the respective liquid, which can facilitate a cleaning interaction between the mixture and the walls of the lumen. In certain embodiments, the liquid-powder mixture comprises mixture of sodium bicarbonate powder and water, where the sodium-bicarbonate is present in an amount greater than the respective saturation level. For example, in a number of embodiments, sodium-bicarbonate can, at certain stages, be present in an amount greater than 10% of the mixture by mass. It has been determined that a mixture of sodium bicarbonate and water can be particularly effective in the disclosed application. Moreover, these constituent components are readily available. However, as noted elsewhere herein, the techniques presented here are not limited to use of a mixture of sodium bicarbonate and water and, as such, it would be appreciated that any suitable liquid-powder mixture can be implemented in accordance with embodiments of the invention.

In some embodiments presented herein, the powder in the mixture is present in an amount below the respective saturation of the associated liquid. However, the liquid is delivered to the target lumen prior to the complete dissolution of the powder in the liquid. In this way, the undissolved powder can still interact with the target lumen to be cleaned.

Moreover, it is to be appreciated that the liquid-powder mixture can be created/obtained in any of a variety of ways in accordance with embodiments of the invention. For example, in certain embodiments, a powder is obtained from a cartridge or other consumable chamber/container, water is obtained from a tap, and these constituent components are mixed within a holding chamber (or within the consumable chamber itself) proximate (e.g., within days or weeks) to the time of cleaning. This approach may be advantageous insofar as powders such as sodium bicarbonate can be relatively stable and can have a long shelf life and suitable sources of water are readily available. However, in other embodiments, the mixture may be obtained in an already mixed form.

As noted, method 240 involves apportioning the liquid-powder mixture into a suitable amount. As illustrated, the apportioned amounts are subsequently delivered through a lumen to be cleaned. Delivering discrete amounts of the mixture can be advantageous insofar as the discrete amounts can be delivered periodically at suitable velocities, and the periodic application of the composition can help facilitate the cleaning of the lumen while not clogging/blocking the target lumens. Moreover, the discrete nature of the delivered amounts can facilitate the maintenance of a suitable delivery velocity, which can also aid cleaning. For example, if the liquid-powder mixture was delivered continuously (and not in discrete, apportioned amounts), this approach might risk ‘clogging’ or otherwise obstructing the lumen so as to reduce the velocity at which the contaminant-detaching fluidic composition flows through the lumen, and can thereby impact cleaning efficacy.

Notably, different amounts of liquid-powder mixture may be differently suitable for the different characteristics of lumens to be cleaned. For example, air/water channels within an endoscope are typically amongst the narrowest lumens and, accordingly, may be more suitably cleaned with relatively smaller amounts of a liquid-powder mixture (whereas using larger amounts of a liquid-powder mixture may result in blocking such a narrow channel). In contrast, the suction/biopsy channels of an endoscope are typically amongst the widest lumens and, accordingly, may be more suitably cleaned with relatively larger amounts of liquid-powder mixture. As such, the amount of liquid-powder mixture apportioned for use in cleaning a given lumen is a function of the geometry of the lumen to be cleaned. It should of course be appreciated that the amount of liquid-powder mixture apportioned can also or alternatively be a function of any of a variety of parameters, including those that relate to the target lumen, in accordance with embodiments of the invention.

The apportioned amount of the liquid-powder mixture can be determined in any of a variety of ways. For example, in certain embodiments, a valve may be used to draw a target amount of liquid-powder mixture from a reservoir. In some embodiments, a self-regulating pressurized system is used to draw a suitable amount of liquid-powder mixture from the reservoir.

As noted, method 240 of FIG. 2A further includes delivering the apportioned amount of liquid-powder mixture through at least a portion of the lumen to be cleaned. In general, a carrier fluid (e.g., air, water, etc.) is used to deliver (e.g., propel) the apportioned amount of the liquid-powder mixture through at least a portion of the lumen to be cleaned at a suitable velocity. The apportioned amount of the liquid-powder mixture is delivered in a manner (e.g., suitable size, suitable velocity, etc.) to provide an appropriate physical interaction between the mixture and the walls of the lumen, meaning that the undissolved powder will physically contact or run against the walls of the lumen to remove contaminants (e.g., bioburdens) therefrom. Of course, the apportioned amount of the liquid-powder mixture may be delivered through the lumen in any suitable way to enable cleaning of lumen in accordance with embodiments of the invention.

Notably, method 240 can be iterated any number of times to facilitate the cleaning of the lumen of a medical device. For example, FIG. 2B illustrates the delivery of one cleaning slug 248 (e.g., an apportioned amount of a liquid-powder mixture) through a lumen 252 to remove contaminants from the walls of the lumen, where the general direction of travel of the slug 248 is represented by arrows 261. That is, as shown, the lumen 252 has one or more contaminants 254 (e.g., bioburdens) disposed on the inner surface/walls 256 of the lumen. It is further illustrated that the cleaning slug 248 is delivered through the lumen 252 to physically interact with the walls of the lumen and thereby remove the contaminants 254 therefrom. The cleaning slug 248 can be considered to be entrained within a carrier fluid, which in this example comprises air (represented by arrows 263).

In general, cleaning slugs presented herein, such as cleaning slug 248, can have different forms/arrangements. For example, in certain embodiments, a cleaning slug presented herein can be a relatively singular/unitary mass (e.g., potentially substantially occluding the lumen while traveling therethrough), which is sometimes referred to herein as a “unitary slug.” However, in other embodiments, a cleaning slug can be an “agglomeration” or “cluster” of smaller masses/groups that travel through the lumen as a loose group (e.g., potentially not occluding the lumen while traveling therethrough), sometimes referred to herein as a “cluster slug.” FIG. 2B schematically illustrates an example in which the slugs 248 are cluster slugs.

In certain embodiments, a cleaning slug can transition between different forms during the slug's life cycle. For example, a slug could be apportioned (initially created) as a unitary slug, but then transition to a cluster slug. This transition could occur before entering the lumen (e.g., in a delivery chamber) and/or while traveling through the lumen.

As noted above, FIG. 2B generally illustrates the delivery of a cleaning slug 248 through a lumen 252. In certain examples, FIG. 2B represents a first stage/phase of a cleaning process, while FIG. 2C represents a second stage/phase of the cleaning process. More specifically, after a cleaning slug 248 is delivered through the lumen 252 (as in FIG. 2B), a fluid flow is delivered through the lumen 252 without any slugs. In the example of FIG. 2C, the fluid flow is comprised of water 265, where the general direction of travel is again represented by arrows 261. In certain examples, the fluid flow (e.g., water 265) is configured to remove residuals 247 from the lumen. The residuals 247 can comprise, for example, some remaining portion of the contaminant 254 and/or portions of the slugs 248 that may remain on the walls of the lumens 252 after passage of the slugs (e.g., the slug can break up into different clusters, some of which remain on the walls of the lumen 252). If present, portions of the slug 248 that may remain on the walls of the lumens 252 may aid in the cleaning process as these portions are flushed through the lumen 252 by the fluid flow.

FIGS. 2B and 2C generally illustrate an arrangement in which the second stage (fluid flow) is interspersed between the delivery of cleaning slugs. That is, in the embodiments of FIGS. 2B and 2C, the delivery of each cleaning slug is followed by a fluid-only flow. In certain alternative embodiments, multiple slugs could alternatively be delivered through a lumen either simultaneously or sequentially, without separation (e.g., without a fluid-only flow).

While FIG. 2B illustrates the delivery of one cleaning slug 248, it should be appreciated that any number of cleaning slugs may be delivered through the lumen in different embodiments. In general, the use of a series of discrete/individual cleaning slugs 248, as opposed to a single large flow, can allow the individual cleaning slugs to maintain sufficient kinetic energy to pass through the lumens at a rate that allows the particles with the slugs to advantageously interact with, and remove contaminants from, the lumen walls.

As noted above, a lumen cleaning process, such as described above with reference to FIGS. 2A, 2B, and 2C, can be implemented in a number of different manners with a number of different lumens. For context, one specific example implementation is described with reference to cleaning at least part of the endoscope 100 of FIG. 1A.

More specifically, in one example cleaning process/cycle, one (1) cleaning slug is fired/shot into the water-jet channel 128 via water-jet connector 138, nine (9) cleaning slugs are then fired into the biopsy/suction channel 122 via suction connector 137, one (1) cleaning slug is then fired into the water-jet channel 128 via water-jet connector 138, three (3) cleaning slugs are then fired into the distal section 122B of the biopsy/suction channel 122 via biopsy valve 128, one (1) cleaning slug is then fired into the water-jet channel 128 via water-jet connector 138, and then nine (9) cleaning slugs are fired into the biopsy/suction channel 122 via suction connector 137. The cleaning cycle can further include firing/shooting six (6) cleaning slugs into the air channel 124 via air connector 143 and firing six (6) cleaning slugs into the water channel 126 via water connector 141 (e.g., in parallel). The firing of the cleaning slugs within each target lumen can be followed by a fluid flow, as described above with reference to FIG. 2C. The cleaning slugs and fluid flows can be delivered via one, or possible multiple connectors (e.g., one connector for the air pipe and one connector for the air/water bottle).

In certain examples, approximately 180-200 grams of a slurry could be used to clean a typical flexible GI endoscope. For example, approximately use 80-100 grams can be used to clean a relatively large channel (e.g., suction/biopsy channel 122) with 21 shots in total and an approximately 15 second delay between each shot. For a relatively small channel (e.g., air/water channels), the process can use approximately 60-80 grams with 12 shots in total and an approximately 30 second delay between each shot. For other small channels (e.g., water-jet channel 128), the process can use approximately 10-20 grams with 3 shots in total and an approximately 30 second delay between each shot. Again, each of these channels can also receive a subsequent fluid flow (e.g., after each cleaning slug), as described above with reference to FIG. 2C.

As noted above, cleaning slugs are delivered to a target lumen with a velocity that is suitable/sufficient to remove contaminants from the walls of the target lumen. The velocity of the cleaning slugs can vary, for example, based on the attributes of the target lumen, the attributes of the of the contaminant-detaching fluidic composition (slurry) used to form the slug, etc. In one illustrative example, the slug velocity for a relatively large lumen may be around 1000 mm/second.

In addition, the cleaning slugs can be delivered within specific pressure and fluid flow (air) ranges. In certain examples, the cleaning slugs can be delivered with a pressure up to approximately 26 psi (air, note this is regulated by a PPR as described below), up to approximately 24 psi (water), etc. Example air flow metrics can include approximately 50 SLPM (large channel no load), approximately 11-17 SLPM (large channel during dosing), approximately 7-10 SLPM (large channel during full load), approximately 5-7 SLPM (small channel no load), and approximately 0.1 SLPM (small channel during full load). It is to be appreciated that these ranges and values are merely illustrative and that aspects of the techniques presented herein are in no way limited to these specific ranges and values.

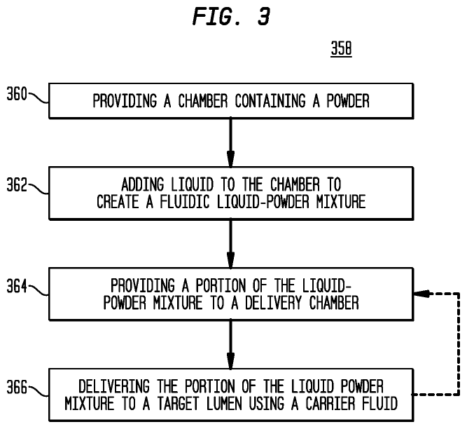

FIG. 3 illustrates another method 358 for cleaning a target lumen in accordance with embodiments of the invention. As shown, method 358 begins at 360 with the providing of a holding chamber containing a powder. This can be accomplished in any of a variety of ways. For example, in some embodiments, a dedicated system for performing the cleaning includes a ‘durable’ chamber configured to receive powder, e.g., via a cartridge, and the providing may thereby be achieved. Such a chamber may be considered to be ‘durable’ insofar as it is intended to be operable for the lifetime of the system. In certain embodiments, a dedicated system for performing the cleaning is configured to receive a disposable/consumable chamber that inherently includes the powder, and in this way, the providing may thereby be achieved. Such disposable/consumable chambers may be provided with sufficient powder to enable multiple cleaning cycles, after which they are ‘consumed’ (depleted). Thereafter, users can obtain additional disposable/consumable chambers that inherently include the powder.

The method 358 further includes, at 362, adding liquid to the holding chamber to create a fluidic liquid-powder mixture. In certain embodiments, the liquid can come from a liquid source that is dedicated to servicing only the holding chamber. In further embodiments, a liquid source is used both to provide liquid to the holding chamber and to provide liquid to act as a carrier fluid. Such a configuration can enable for a more efficient design.

The method 358 further includes, at 364, providing a portion of the liquid-powder mixture to a delivery chamber. As indicated previously, this can be achieved in any of a variety of ways. For example, a valve may be used to provide a portion of the liquid-powder mixture to the delivery chamber. In certain embodiments, the size of the portion of the of the liquid-powder mixture provided to the delivery chamber is a function of characteristics of the target lumen to be cleaned. This aspect is described further below.

The method 358 further includes, at 366, delivering the portion of the fluidic liquid-powder mixture to a target lumen using a carrier fluid. In effect, the fluidic liquid-powder mixture can be made to interact with the lumen (akin to ‘brushing’ it). As illustrated, this providing of fluidic liquid-powder mixture to the delivery chamber and the subsequent delivery can be iterated a plurality of times to effectuate the cleaning of the lumen.

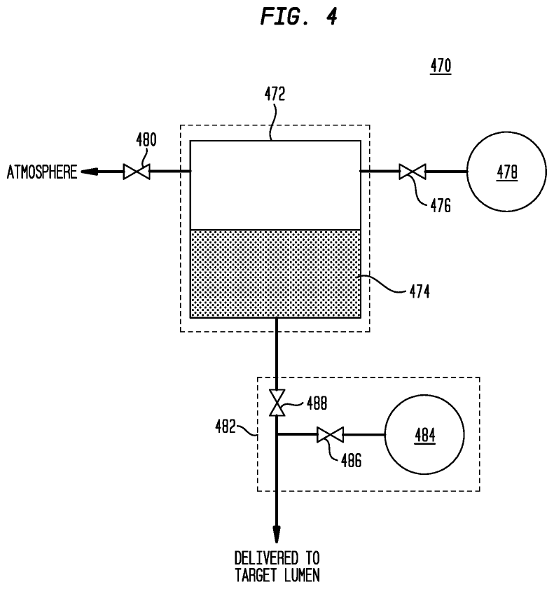

It is to be appreciated that systems for cleaning a lumen of a medical device, in accordance with embodiments presented herein, can take any of a number of different forms/arrangements. However, in general, the systems include: a holding chamber for creating/housing a powder and/or liquid-powder mixture and a mechanism for delivering a portion of the liquid-powder mixture to the target lumen. FIGS. 4, 5, and 6 illustrate various aspects of example systems that can be implemented in accordance with embodiments presented herein.

Referring first to FIG. 4, shown is a system 470 for cleaning a lumen of a medical device using a liquid-powder mixture, in accordance with embodiments presented herein. More specifically, the system 470 includes a holding chamber 472 for creating/housing a liquid-powder mixture 474. In the illustrated embodiment, the holding chamber 472 is provided with the powder that is used to form the liquid-powder mixture. For example, the holding chamber 472 may be a consumable component of the system 470 and may be replaced when its contents have been used. The holding chamber 472 interfaces with an inlet valve 476 for receiving liquid from a liquid source 478. A relief valve 480 can be used to relieve pressure created during creation of the mixture. As can be appreciated, the liquid-powder mixture 474 can be created using any suitable constituent components. For example, in certain embodiments, the powder that is provided with the holding chamber 472 is sodium bicarbonate, and the liquid source 478 is a source for water. Of course, it can be appreciated that the holding chamber 472 can receive liquid and powder in any of a variety of ways in accordance with embodiments of the invention. For example, in some embodiments, the chamber is configured to receive powder from a powder reservoir, e.g., a sodium bicarbonate cartridge. In some embodiments, a pump is used to provide liquid to the chamber in lieu of directly using a valve to do so. In some embodiments, the holding chamber 472 may include mechanisms (not illustrated) for facilitating the mixing of the received powder and liquid. For example, a stirring mechanism or an agitation mechanism may be implemented to facilitate mixing.

The system 470 further includes a delivery mechanism 482 for delivering a portion of the liquid-powder mixture to the target lumen. In the illustrated embodiment, the delivery mechanism is in the form of an aggregate of a carrier fluid source 484, a first valve 486, and a second valve 488. As can be appreciated, the carrier fluid 484 may be made to flow through the target lumen via the valve 486, and portions of the liquid-powder mixture may be entrained within this flow. It should be noted that any suitable carrier fluid may be implemented, for example, the carrier fluid source may comprise at least one of: air, water, ethanol, nitrogen, and carbon dioxide. In the illustrated embodiment, a valve 420 is configured to implement the amount of liquid-powder mixture that is entrained within the carrier fluid. For example, valve 482 may be open to the holding chamber 472, and the holding chamber 472 may be pressurized via the liquid source 478 and valve 476 thereby resulting in delivery of a portion of the liquid-powder mixture to the delivery mechanism 482. Of course, it should be appreciated that any suitable mechanism for implementing the amount to be entrained within the carrier fluid may be applied in accordance with embodiments of the present invention.

While one system architecture for cleaning a medical device having a lumen has been illustrated, it should be appreciated that the described concepts can be implemented in any of a variety of ways in accordance with embodiments of the invention. For example, in some embodiments, the carrier fluid source is additionally used to create the liquid-powder mixture and, as such, a separate liquid source (e.g., 478) may not be necessary. In some embodiments, a selectable plurality of carrier fluid sources can be implemented. Thus, for instance, in some embodiments, a source of air and a source of water can each supply carrier fluid for delivery of liquid-powder mixture to the lumen and the water source may further be used to facilitate the creation of the liquid-powder mixture. In some embodiments, the chamber may include a discrete pressure source to facilitate the delivery of a portion of the liquid-powder mixture to the delivery mechanism, such that the liquid source does not have to facilitate said delivery.

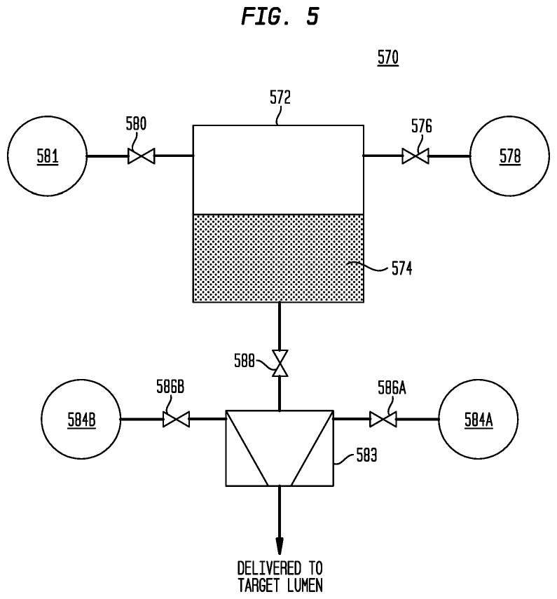

In certain embodiments, a separate chamber is implemented to facilitate the propulsion of the mixture through the lumen to be cleaned. For example, FIG. 5 illustrates a system 570 that includes a holding chamber 572 for creation/housing of a liquid-powder mixture 574 and a delivery chamber 583 for developing the velocity of the liquid-powder mixture for subsequent delivery through the lumen. In the illustrated embodiment, a powder source 581 is coupled to the holding chamber 572 via a valve 580, and a liquid source 578 is coupled to the holding chamber 572 via a valve 576. The powder source 581 may be, for example, a cartridge and the liquid source 578 may be, for example, a pressure-regulated mains water.

As noted, the system 570 also comprises the delivery chamber 583 for delivery of a portion of the liquid-powder chamber to the target lumen to be cleaned.

As shown, the delivery chamber 583 is coupled to each of two carrier fluid sources 584A and 584B via respective valves 586A and 586B. For example, air and water may serve as carrier fluids for the illustrated system. The amount of liquid-powder mixture to be entrained in the carrier fluid can be controlled by a valve 588.

In general, one example purpose of a delivery chamber presented herein, such as delivery chamber 583, is to create an air gap between the slug source (holding chamber 572) and the target lumen to be cleaned. The creation of the air gap provides a location (e.g., the delivery chamber 583) where the cleaning slugs can be accelerated so as to enter the target lumen at a suitable (e.g., selected) velocity. That is, the delivery chamber 583 provides a region where the system 570 uses one or more fluids (e.g., air and/or water) to accelerate the cleaning slugs. Without the delivery chamber 583, the cleaning slugs would enter the target lumen with the same speed at which the cleaning slugs exit the holding chamber 571, which is likely too slow to effectively clean the target lumen (e.g., delivery chamber 583, enables the system 570 to provide sufficient kinetic energy to propel the slugs through the entire lumen at a desired rate).

As shown in FIG. 5, the delivery chamber 582 defines a frustoconical shape, which can be beneficial in a number of respects. For example, such a geometry can aid the flow of the ‘slurry,’ e.g., directing it towards the target lumen. Additionally, the frustoconical shape can cause the development of a “vortex” of the carrier fluid within the delivery chamber 582.

It can be appreciated that while a certain configuration has been illustrated, systems implementing a discrete delivery chamber can be implemented in any of a variety of ways in accordance with embodiments of the invention. For example, in some embodiments, the delivery chamber is coupled to only a single carrier fluid source.

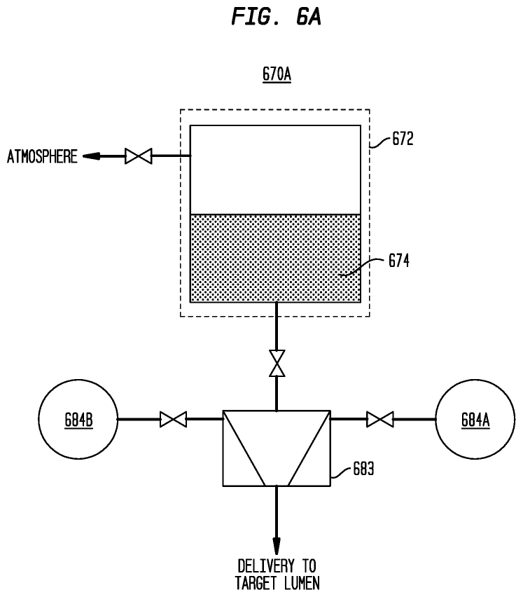

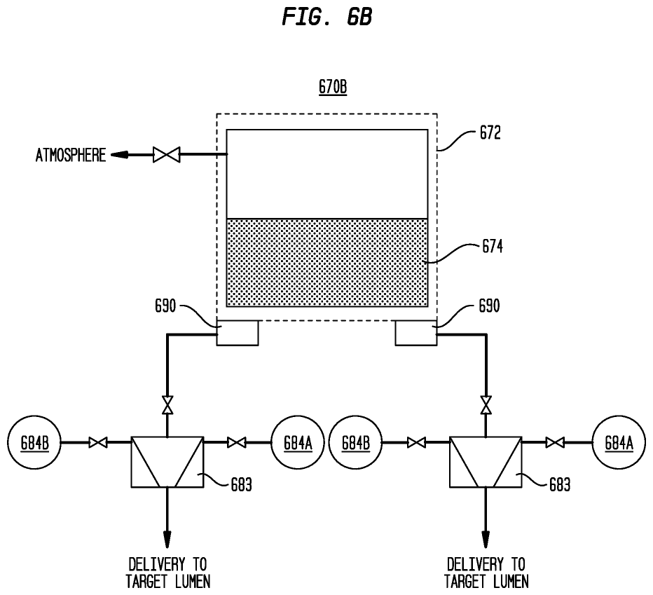

While the embodiment illustrated in FIG. 5 depicts an architecture whereby powder may be provided to a chamber via, for example, a cartridge, in some embodiments the chamber may be a consumable component, as mentioned previously. Accordingly, FIG. 6A illustrates a system 670A for cleaning a lumen of a medical device with a consumable component and a delivery chamber.

In particular, the system 670A includes a holding chamber 672 in the form of a consumable component that is provided with powder. A liquid-powder mixture 674 can be created/housed within the holding chamber 672 using liquid from carrier fluid source 684A. The system 670A further includes a carrier fluid source 684B that may house a gaseous carrier fluid. Similar to the system 570 of FIG. 5, the system 670A further includes a delivery chamber 683 operable to deliver the liquid-powder mixture to the lumen for cleaning. In certain embodiments, a pump 690 is also provided between the holding chamber 672 and the delivery chamber 683.

The use of a chamber in the form of a consumable component, as shown in FIG. 6A, may be advantageous insofar as it simplifies the design and enhances user-operability. For example, the use of such a configuration can eliminate the need for a discrete powder handling mechanism. Although the illustrated embodiments depicts a chamber that houses powder that is subsequently hydrated, in some embodiments a chamber is provided with a pre-made liquid-powder mixture. For example, a chamber that houses a powder that is insoluble in a corresponding liquid may be implemented. The insolubility of the powder in the respective liquid may allow the chamber to have a suitable shelf life, and therefore may be commercially viable.

FIG. 6B illustrates another system 670B for cleaning a lumen of a medical device with a consumable component and a delivery chamber. System 670B is similar to system 670A of FIG. 6A and also includes a holding chamber 672 in the form of a consumable component that is provided with powder, where a liquid-powder mixture 674 can be created/housed within the holding chamber 672 using liquid from carrier fluid source 684A. The system 670B further includes a carrier fluid source 684B that may house a gaseous carrier fluid.

However, unlike system 670A of FIG. 6A, the system 670B includes two delivery chambers 683 each operable to deliver the liquid-powder mixture to a target lumen for cleaning.

Also shown in FIG. 6B are two pumps 690 provided between the holding chamber 672 and a corresponding delivery chamber 683. In certain examples, the system 670B can be used to concurrently (e.g., simultaneously, sequentially, etc.), clean two lumens.

In certain embodiments, a system presented herein further includes at least one distribution manifold that may couple to a plurality of ports/channels/lumens of the medical device to be cleaned. In several embodiments, a single delivery chamber is coupled to a single port of a medical device. Each of multiple delivery chambers may be simultaneously coupled to discrete ports of a single medical device. Similarly, in some embodiments, a system may include a holding chamber and a plurality of delivery chambers, the holding chamber may provide cleaning slugs to each of the plurality of the delivery chambers.

In certain embodiments of the invention, consumable chambers that house constituent components used for cleaning for use in systems for cleaning medical devices having lumens (e.g., those described elsewhere herein) are implemented. In this context, ‘consumable chambers’ can be understood to be those that are not intended to be permanent fixtures of the systems with which they interact with. For instance, such a consumable chamber can be obtained, made to interface with a respective cleaning system, and once its constituent components within it have been used up by the cleaning system, they may be disposed of or else sent to a center for reprocessing. Subsequently, a user can obtain another consumable chamber where further cleaning is required. The use of such ‘consumable chambers’ can greatly enhance the efficiency and operability of the disclosed cleaning systems.

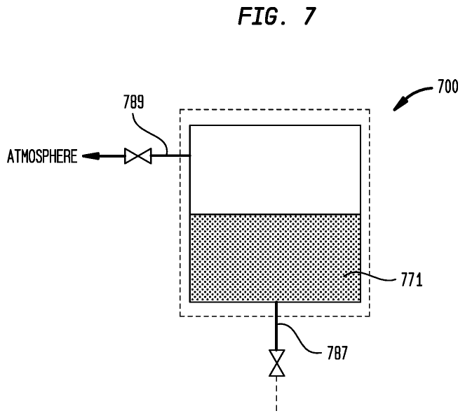

Accordingly, FIG. 7 illustrates a consumable holding chamber 772 housing at least one constituent component for use in a system for cleaning a lumen of a medical device, in accordance with embodiments of the present invention. In particular, it is illustrated that the consumable holding chamber 772 includes at least one constituent component 771 for use in a cleaning system (e.g., such as any of those described above). For example, in certain embodiments, the consumable holding chamber 772 is provided with sodium bicarbonate and the cleaning system that interfaces with the consumable chamber may thereafter provide the holding chamber 772 with water to create a sodium bicarbonate-water mixture that can be used for cleaning. In some embodiments, the consumable holding chamber 772 inherently includes a liquid-powder mixture (e.g., prior to interfacing with a respective cleaning system). For example, certain liquid-powder mixture combinations may have a more durable shelf life (e.g., as compared to the mixture of sodium bicarbonate and water), and therefore it may be more commercially viable for consumable chambers to include such liquid-water mixtures. It is further illustrated that the consumable holding chamber 772 includes two interfaces 787, 789 for engaging with a cleaning system. For instance, interface 787 can allow the cleaning system to provide liquid to holding chamber 772 via a valve to create the liquid-powder mixture used for cleaning. The interface 787 may additionally/alternatively allow the cleaning system to draw a portion of the liquid-powder mixture from the chamber for subsequent delivery to a target lumen. The illustrated consumable chamber further includes interface 789 for engaging with a cleaning system. In particular, it is illustrated that 789 allows the consumable chamber to interface with a relief valve, which can help direct the fluid flow to/from the consumable chamber.

It should be appreciated that while a specific configuration for a consumable chamber has been illustrated, embodiments of the invention can be implemented in any of a variety of ways in accordance with embodiments of the invention. For example, in some embodiments, a third interface is included for engagement with a dedicated liquid source for creatin the liquid-powder mixture. In some embodiments, respective valves may be integrated with the consumable chamber. In general, the disclosed concepts can be implemented in any of a variety of ways in accordance with embodiments of the invention.

In certain embodiments, a method of cleaning a lumen of a medical device includes determining the fluidic resistance/impedance (and/or conductance) of the target lumen to be cleaned and using the determined fluidic resistance to inform the cleaning methodology. For example, different lumens may have different characteristics such as geometry, etc., and enhancing the cleaning efficacy/efficiency may be a function of these particular characteristics. Fluidic resistance may be a suitable indicator of these characteristics. Generally, fluidic resistance can be understood to relate to how much a lumen restricts flow.

For example, determining the fluidic resistance of the target lumen of the medical device can include flowing a fluid comprising a known specific gravity through the target lumen of the medical device and measuring a flow rate and/or a pressure differential of the fluid being flowed through the target lumen of the medical device. These parameters can then be used to compute the fluidic resistance of the target lumen. Such a method is merely illustrative and other techniques could alternatively be used to determine the fluidic resistance of a target lumen (e.g., determine the fluidic resistance directly from the known dimensions of a target lumen).

As noted, the fluidic resistance of the target lumen may be used to control the apportionment of the liquid-powder mixture and/or the delivering of the apportioned amount. For example, in one arrangement, a suction/biopsy channel of an endoscope is the target lumen to be cleaned. Suction/biopsy channels are relatively larger lumens, and the dimensions of the channel may be used to establish a relatively larger size of the apportioned amount. Conversely, air-water channels of an endoscope relatively smaller lumens, and the dimensions of these channel may be used to establish a relatively smaller size of the apportioned amount.

In certain examples, the fluidic resistance of the target lumen can be used (e.g., periodically, continually, etc.) to update cleaning parameters (e.g., in real-time) as appropriate to enhance cleaning efficacy. For example, the frequency of the delivery of apportioned amounts can be informed by the determined fluidic resistance. More details around the technique of determining the fluidic resistance of the target lumen can be found in Australian Patent Application No. 2021901734, entitled “Systems and Methods for the Identification, Evaluation, and/or Closed-Loop Cleaning of lumens,” filed Jun. 9, 2021, the content of which is hereby incorporated by reference herein, and concurrently filed patent application entitled “Systems and Methods for the Identification, Evaluation, and/or Closed-Loop Reprocessing of Lumens,” the content of which is also hereby incorporated by reference herein.

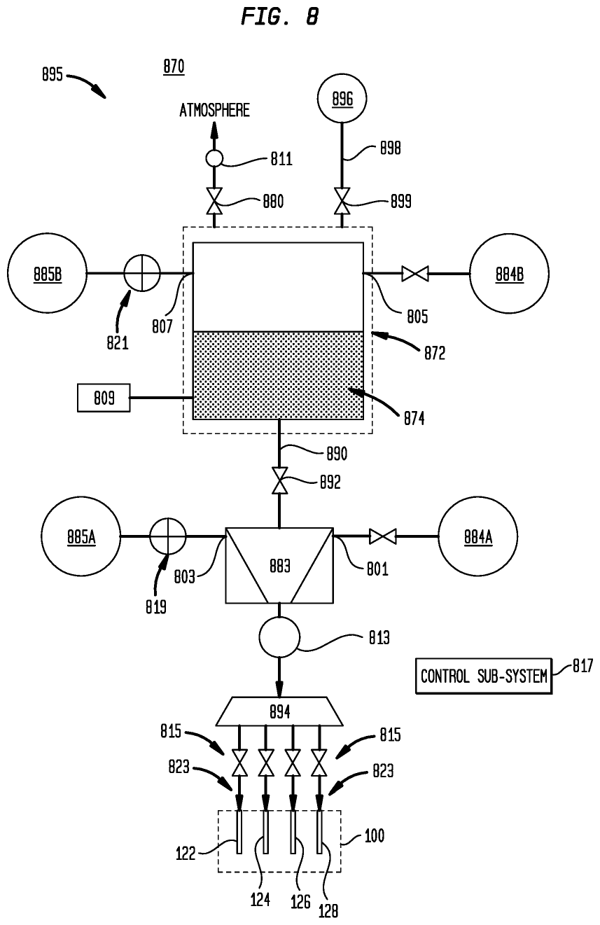

It is to be appreciated that the above-described concepts can be applied in any of a variety of ways in accordance with embodiments of the invention. However, FIG. 8 illustrates one example system 870 for cleaning a lumen that can incorporate specific elements of the above-described concepts, in accordance with embodiments of the present invention. For ease of description, system 870 will generally be described with reference to endoscope 100 of FIG. 1.

More specifically, the system 870 includes a control sub-system 817, a holding sub-system 895, and a delivery sub-system 897. The holding sub-system 895 includes, among other elements, a holding chamber 872 for mixing a powder and liquid to form a liquid-powder mixture 874. The delivery sub-system 897 comprises, among other elements, a delivery chamber 883 for creating a flow of fluid to propel the cleaning slug through at least a portion of a channel, such as channels 122, 124, 126, or 128, of endoscope 100. In the illustrated embodiment of FIG. 8, the delivery chamber 883 includes an internal volume that can be characterized as having a frustoconical-like shape such that entry of fluid flow from one or more sides will create a flow of the fluid that increases in velocity as the flow approaches the narrow end of the frustum. However, it is to be appreciated that the use of a frustoconical shape, although potentially advantageous, is merely illustrative and that a delivery chamber can have any suitable geometry in accordance with embodiments of the invention. For example, in some embodiments a delivery chamber can have a cylindrical form factor. In several embodiments, a delivery chamber can be characterized as having a hemispherical shape.

Returning to the example of FIG. 8, a slurry conduit 889 fluidly connects the holding chamber 872 to the delivery chamber 883 and a slurry valve 892 is preferably provided in the slurry conduit. Of course, it should be appreciated that any suitable configuration that allows for a liquid-powder slurry to be developed, apportioned into a cleaning slug, and delivered at a suitable velocity to the lumen of a medical device can be implemented in accordance with embodiments of the invention.

In the illustrated embodiment of FIG. 8, the delivery chamber 883 is, in turn, fluidly connectable to at least one of the channels (e.g., 122, 124, 126, or 128) of the endoscope 100. In the illustrated embodiment, a distribution manifold 894 is provided between the delivery chamber and the endoscope channels so that a channel, or portion of a channel, can be selected for cleaning. In some embodiments, a delivery chamber can be associated with a single port of a medical device. In other embodiments, one holding chamber provides slurry to each of several delivery chambers, and each of the several delivery chambers are each associated with a single port of a medical device. Of course, it should be appreciated that any suitable configuration that allows a cleaning slug of liquid-powder slurry to be delivered at a suitable velocity through the lumen of a medical device can be implemented in accordance with embodiments of the invention.

In the illustrated embodiment of FIG. 8, the powder is provided to the holding chamber from a powder cartridge 896 via a powder conduit 898. A powder valve 899 is positioned in the powder conduit 898 upstream of the holding chamber 872 inlet to seal the holding chamber from the powder cartridge, when required. The holding chamber 872 also includes a relief valve 880 to allow the escape of any entrapped air during liquid filling.

Of course, it can be appreciated from the above description, powder can be provided in any suitable way to form the slurry in accordance with embodiments of the invention. For example, in some embodiments, the powder cartridge may be omitted, and the powder required for the cleaning process is simply placed into the holding chamber ready for use. In a further not shown embodiment, the powder for one complete cleaning cycle is placed in the holding chamber in a pierceable powder pod.

In the illustrated embodiment, the delivery chamber 883 includes a primary liquid port 801 and a primary gas port 803 for respectively allowing the entry of a liquid from a primary liquid supply 884A and a gas from gas supply 885A. Similarly, the holding chamber 872 includes a secondary liquid port 805 and a secondary gas port 807, which feed from liquid supply 844B and gas supply 885B respectively. In the illustrated embodiment, a vibration motor 809 is provided and positioned proximate the exit of the holding chamber 872 exit for promoting the egress of slurry or assisting in the mixing process, if/when needed.

In addition to the above, the system 870 of FIG. 8 includes optical sensors 811 and pressure sensors 813, as discussed more specifically below, for monitoring the operation of the slurry production and cleaning process. For example, the pressure sensors 813 may cooperate to detect if the endoscope 100 is connected to the system and sense if there are any blockages in the system. In those circumstances, a fault condition will be generated by the control sub-system 817 (e.g., a computer control (not shown) for programmable control of the operation of the various control valves, motors and/or other pumping systems in accordance with the method of the proposed embodiment of the invention). The control sub-system 817, which can be integrated with the system 870 or a separate computing device, can enable the system 870 to be programmed to clean the various endoscopes, or other medical devices, available on the market to a sufficient degree to adhere to the various regulatory authority and in an enhanced time to shorten the downtime of the device. The user would simply connect the system to the endoscope and recall the required cleaning program. Of course, as can be appreciated, any suitable sensors and control sub-systems can be implemented to affect the operation of cleaning systems in accordance with embodiments of the present invention.

Turning specifically now to operation of the system 870 of FIG. 8, a first step according to the illustrated embodiment in FIG. 8 may be that one or more target channels of endoscope 100 are flushed with water and or a combination of gas and water. This may be achieved, for example, by first closing the slurry valve 892. A flow of gas, such as compressed air, and water are then fed into the delivery chamber 883 from the primary liquid port 801 and gas port 803. The mix of air and water then enter each of the internal channels through the distribution manifold 894 to exit through an exit point in the endoscope. It can be appreciated that the channels may be flushed sequentially at some phases of the flushing cycle and/or the channels may be flushed simultaneously at some phases of the flushing cycle. In other embodiments, the step of flushing is omitted, and the process starts with the first substantive cleaning step below.

As noted, a step in the cleaning process is the obtaining (e.g., the formation) of the slurry mix. In the example of FIG. 8, the slurry mix is formed by providing an amount of powder from the powder cartridge 896, as well as an appropriate amount of a liquid, into the holding chamber 872, thereby creating the slurry mix 874. The liquid may be provided into the holding chamber 872 from the primary liquid port 801 in the delivery chamber 883 feeding up to the holding chamber 872 after opening the slurry valve, or directly from the secondary liquid port 805. In operation, the powder is mixed with the liquid to create the slurry 874. The slurry mix 874 may form naturally upon introduction of the liquid to holding chamber, or alternatively, the vibration motor 809 may be activated to ensure the slurry is mixed to the desired level. It is noted that it is not proposed that all the powder is dissolved in the liquid. To this end, the non-dissolved powder assists the cleaning function.

FIG. 8 is described with reference to an embodiment in which the powder is sodium bicarbonate, and the liquid is water. However, other powders may be used to create the slurry without departing from the scope of the invention. Similarly, other liquids may be used besides water without departing from the scope of the invention.

It is to be appreciated that the slurry (liquid-powder mixture) 874 can be created in a variety of ways. For example, in one method all the control valves 815 at the output of the distribution manifold 894 are initially closed and no gas is being provided to any of the chambers. The relief valve 880, the slurry valve 892, and the primary liquid port 801 are then opened allowing water to fill the delivery chamber 883. When the delivery chamber is filled, water goes into the holding chamber 872 through the slurry conduit 889 and hydrates the powder from bottom. This way, a uniform slurry is formed without the need to use the vibration motor 809. At some point during the filling of the delivery chamber 883, the secondary liquid port 805 may also open to fill the holding chamber 872 faster, if required. Once the holding chamber is appropriately filled with water, as determined for example by an optical sensor 811 at the relief valve, the primary and secondary liquid ports close.

The operations of system 870 also include apportioning the slurry 874 and forming a cleaning slug. In one specific example, the control sub-system 817 (e.g., computing device) closes the slurry valve 892 and opens at least one of the control valves 815. The control sub-system 817 then commands the system 870 to create a positive pressure difference between the holding chamber 872 and the delivery chamber 883 and then open the slurry valve 892. The created positive pressure difference pushes a flow of slurry, already created in the holding chamber 872, into the delivery chamber 883. The slurry valve 892 then closes and stops the flow of slurry from holding chamber 872 into the delivery chamber 883 and a cleaning slug of slurry is therefore defined.

It can be appreciated that creating a pressure difference between the holding chamber 872 and the delivery chamber 883 can be done in variety of ways. For example, the positive pressure difference can be achieved by controlling the pressure of gas or liquid using pumps, pressure regulators, proportional pressure regulators (PPR) or electrical pressure regulators (EPR). For example, in one specific and illustrative arrangement, the gas pressure can be controlled using two proportional pressure regulators, namely a primary PPR 819, and a secondary PPR 821. The primary PPR 819 is positioned between the primary gas supply 885A and the primary gas port 803 and controls the gas pressure in the delivery chamber 883. The secondary PPR 821 is positioned between the secondary gas supply 885B and secondary gas port 807 and controls the gas pressure of the holding chamber 872. The PPRs 819 and 821 are controlled by the automated control to create a selectable positive pressure difference between the holding chamber and the delivery chamber.

As noted, the system 870 delivers the cleaning slug (apportioned amount of the liquid-powder mixture) through at least a portion of a channel of the endoscope 100 to be cleaned. Delivery of a cleaning slug into the endoscope channel can occur in a variety of ways. For example, before the apportioned liquid-powder mixture is transferred into the delivery chamber 883, carrier fluid from primary gas supply 884B is supplied to the delivery chamber 883 at a regulated pressure using primary PPR 819. The carrier gas may create a fluid flow inside the delivery chamber 883 that propels the apportioned liquid-powder mixture into a selected internal channel of endoscope 100 through the distribution manifold 894, which has already selected one of the control valves 815 to be opened at this time (e.g., the cleaning slug is accelerated to the appropriate velocity). Once at the bottom of the delivery chamber 883, the cleaning slug exits the delivery chamber, enters the distribution manifold 894, and then enters a selected internal channel of the endoscope 100 at an appropriate velocity. The process may be repeated many times for an internal channel of the endoscope 100 before moving on to the next internal channel closing one control valve 815 and opening another control valve 815.

Another technique for delivering a cleaning slug of slurry into the target lumen in FIG. 8 uses a self-regulating bi-stable process. In such examples, the carrier fluid from primary gas supply 885B is again supplied to the delivery chamber 883 at a regulated pressure using primary PPR 819. Once the slurry valve 892 opens, a cleaning slug of slurry may transfer into the delivery chamber 883. It can be appreciated that the transfer of the first cleaning slug can happen automatically (e.g., because of gravity) or may be assisted by creation of a positive pressure difference between the holding chamber 872 and the delivery chamber 883. If used, the positive pressure difference may naturally be brought about by the high flow of air through the endoscope due to the low fluidic resistance of an un-occluded lumen. Once this cleaning slug occludes the exit of the delivery chamber, the pressure of the delivery chamber 883 will increase due to the increased fluidic resistance of the fluidic path downstream of the delivery chamber. As a result, the holding chamber 872 and delivery chamber 883 will settle at a similar pressure, which in turn, stops any additional flow of slurry into the delivery chamber. The cleaning slug then proceeds through the selected internal channel to then exit the endoscope 100. When the cleaning slug of slurry exits the endoscope channel, the pressure of the delivery chamber 883 will drop because of the lower fluidic resistance of the downstream fluidic line and the limited flow rate of the carrier gas. This will result in a pressure difference between the delivery chamber 883 and the holding chamber 872. As a result, another cleaning slug of slurry will be drawn from the holding chamber 872. The newly drawn cleaning slug is again acted upon by the gas flow to propel through the delivery chamber 883, distribution manifold 894, and into a selected internal channel of the endoscope 100.

In general, as long as the flow of pressure-regulated gas continues and the supply of slurry in the holding chamber is not finished, the process will essentially continue automatically with the manifold 894 selecting which internal channel will receive the cleaning slug of slurry. More specifically, once the cleaning slug has cleared through an internal channel, the control sub-system can change the states of control valves 815 and the process is repeated for one or more endoscope channels. In a variation of this, the process may be repeated multiple times through the same internal channel until a sufficient level of debris removal is achieved before moving on to another channel.

By selecting the appropriate parameters such as size of the chambers, amount of liquid, amount of powder, and gas pressure, the system can self-regulate and prevent endoscope internal channel blockage while cleaning the endoscope. In some embodiments, the size of the cleaning slug can more deliberately be altered by changing the gas pressure, amount of water, amount of powder, size of the chambers, etc.

In a variation of the above process according to a further embodiment, the slurry valve 892 and all control valves 815 associated with each connector 823, are initially closed. Similar to the previous technique, once the slurry is formed in the holding chamber 872 using one of the earlier mentioned processes, a positive pressure difference is created between the holding chamber and the delivery chamber using primary and secondary proportional pressure regulators (PPRs) or other means such as electrical pressure regulators (EPRs). Then, the slurry valve 892 opens and the positive pressure difference draws a cleaning slug of slurry into the delivery chamber. The slurry valve then closes and stops the flow of slurry from the holding chamber into the delivery chamber to define a cleaning slug of slurry. A pressure-regulated gas, typically compressed air, is then introduced into the delivery chamber 883 through primary gas port 803 to again create a flow of gas. However, unlike the previous method, the cleaning slug is retained in the delivery chamber 883 until one of the control valves 815 in the distribution manifold 894 is opened. After a selectable time, one of the control valves 815 is opened and the cleaning slug is propelled at an appropriate velocity into an endoscope channel using pressurized air. By doing so, the cleaning efficiency may be enhanced by providing a better control over the size of the cleaning slug because the cleaning slug will be fully defined within the delivery chamber before it will be propelled as whole rather than being partially propelled. The described high velocity cleaning slugs of slurry can use, for example, their composition and velocity created by the pressurized gas, water or mixture of gas and water in the delivery chamber, to create a strong physical cleaning action across the internal walls of the selected endoscope channel.

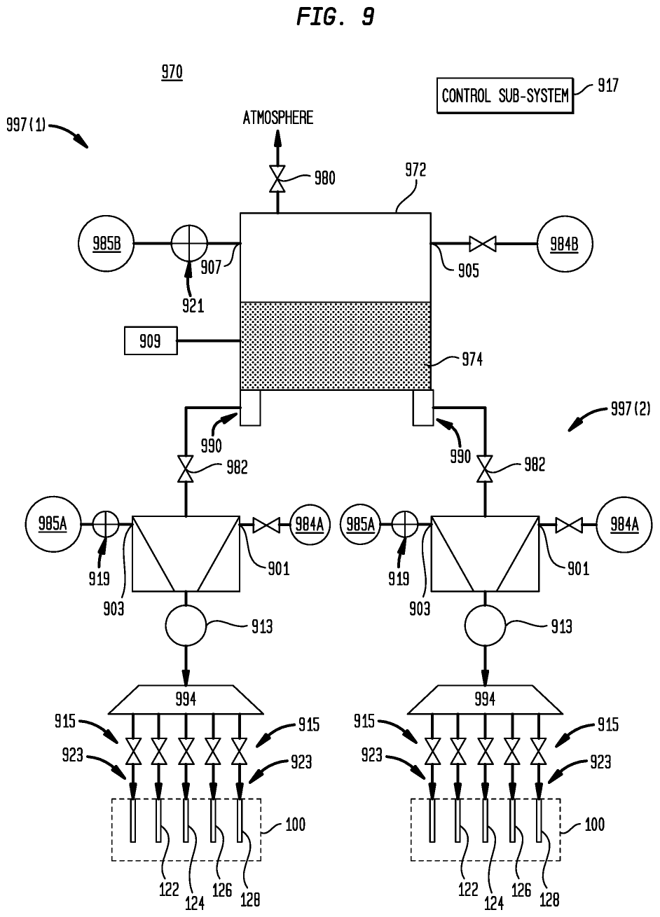

FIG. 9 illustrates another example system 970 for cleaning a lumen that can incorporate specific elements of the above-described concepts, in accordance with embodiments of the present invention. For ease of description, system 970 will generally be described with reference to endoscope 100 of FIG. 1.

More specifically, the system 970 includes a control sub-system 917, a holding sub-system 995, a first delivery sub-system 997(1), and a second delivery sub-system 997(2). The holding sub-system 995 includes, among other elements, a holding chamber 972 for mixing a powder and liquid to form a liquid-powder mixture 974 (e.g., a consumable chamber), while each delivery sub-system 997(1) and 997(2) comprises, among other elements, a delivery chamber 983 for creating a flow of fluid to propel the cleaning slug of slurry through at least a portion of a channel, such as channels 122, 124, 126, or 128, of endoscope 100. In the illustrated embodiment of FIG. 9, the delivery chambers 983 each include an internal volume that can be characterized as having a frustoconical-like shape such that entry of fluid flow from one or more sides will create a flow of the fluid that increases in velocity as the flow approaches the narrow end of the frustum. However, it is to be appreciated that the use of a frustoconical shape, although potentially advantageous, is merely illustrative and that a delivery chamber can have any suitable geometry in accordance with embodiments of the invention. For example, in some embodiments a delivery chamber can have a cylindrical form factor. In several embodiments, a delivery chamber can be characterized as having a hemispherical shape.

As noted above, the holding sub-system 995 comprises a holding chamber 972. In this example, the holding chamber 972 comprises a consumable component initially provided with a powder therein. A liquid is introduced into the holding chamber 972 for mixing with the powder to form the liquid-powder mixture 974. To this end, the holding chamber 972 includes a secondary liquid port 905 and a secondary gas port 907, which feed from liquid supply 984B and gas supply 985B respectively. In the illustrated embodiment, a vibration motor 909 is optionally provided and positioned proximate the exit of the holding chamber 972 exit for promoting the egress of slurry or assisting in the mixing process, if/when needed. The holding chamber 972 can also include a relief valve 980 to allow the escape of any entrapped air during liquid filling.

As noted, the system 970 includes two delivery sub-systems 997(1) and 997(2). In general, the two delivery sub-systems 997(1) and 997(2) are substantially similar and, as such, the following description is provided with reference to the delivery sub-system 997(1). It is to be appreciated that this description applies similarly to delivery sub-system 997(2).

As shown, the delivery sub-system 997(1) comprises a pump 990 and a slurry valve 992 fluidly connecting the holding chamber 972 to the delivery chamber 983. The pump 990 and slurry valve 992 are operable to provide/deliver an apportioned amount of the liquid-powder mixture 974 (a cleaning slug) to the delivery chamber 983. Of course, it should be appreciated that any suitable configuration that allows for a liquid-powder slurry to be developed, apportioned into a cleaning slug, and delivered to a delivery chamber could be used in accordance with embodiments of the invention

In the illustrated embodiment, the delivery chamber 983 includes one or more primary liquid ports 901 and one or more primary gas ports 903 for respectively allowing the entry of a liquid from a primary liquid supply 984A and a gas from gas supply 985A (e.g., there may be plurality of exits with the chambers 983 for each of liquid and gas deliver). The delivery chamber 983 is, in turn, fluidly connectable to at least one of the channels (e.g., 122, 124, 126, 128, etc.) of the endoscope 100. In the illustrated embodiment, a distribution manifold 994 is provided between the delivery chamber and the endoscope channels so that a channel, or portion of a channel, can be selected for cleaning. In some embodiments, a delivery chamber can be associated with a single port of a medical device. In other embodiments, one holding chamber provides slurry to each of several delivery chambers, and each of the several delivery chambers are each associated with a single port of a medical device. Of course, it should be appreciated that any other suitable configuration can be implemented in accordance with embodiments of the invention.

In addition to the above, the delivery sub-system 997(1) includes a pressure sensor 913, as discussed more specifically below, for monitoring the operation of the cleaning process. For example, the pressure sensor 913 may be used to detect if the endoscope 100 is connected to the system and/or to sense if there are any blockages in the system. In those circumstances, a fault condition will be generated by the control sub-system 917 (e.g., a computer control (not shown)) for programmable control of the operation of the various control valves, motors and/or other pumping systems in accordance with the method of the proposed embodiment of the invention). The control sub-system 917, which can be integrated with the system 970 or a separate computing device, can enable the system 970 to be programmed to clean the various endoscopes, or other medical devices, available on the market to a sufficient degree to adhere to the various regulatory authority and in an enhanced time to shorten the downtime of the device. The user would simply connect the system to the endoscope and recall the required cleaning program. Of course, as can be appreciated, any suitable sensors and control sub-systems can be implemented to affect the operation of cleaning systems in accordance with embodiments of the present invention.

Turning specifically now to operation of the system 970 of FIG. 9, a first step according to the illustrated embodiment in FIG. 9 may be that one or more target channels of endoscope 100 are flushed with water and or a combination of gas and water. This may be achieved, for example, by first closing the slurry valve 992. A flow of gas, such as compressed air, and water are then fed into the delivery chamber 983 from the primary liquid port 901 and gas port 903. The mix of air and water then enter each of the internal channels through the distribution manifold 994 to exit through an exit point in the endoscope. It can be appreciated that the channels may be flushed sequentially at some phases of the flushing cycle and/or the channels may be flushed simultaneously at some phases of the flushing cycle. In other embodiments, the step of flushing is omitted, and the process starts with the first substantive cleaning step below.

As noted, a step in the cleaning process is the obtaining (e.g., the formation) of the slurry mix. In the example of FIG. 9, the slurry mix is formed by providing an amount of fluid for mixing with the powder in the holding chamber 972, thereby creating the slurry mix 974. The liquid may be provided into the holding chamber 972 directly from the secondary liquid port 905 (or another liquid line). In operation, the liquid is mixed with the powder to create the slurry 974. The slurry mix 974 may form naturally upon introduction of the liquid to holding chamber, or alternatively, the vibration motor 909 may be activated to ensure the slurry is mixed to the desired level. It is noted that it is not proposed that all the powder is dissolved in the liquid. To this end, the non-dissolved powder assists the cleaning function.

FIG. 9 is described with reference to an embodiment in which the powder is sodium bicarbonate, and the liquid is water. However, other powders may be used to create the slurry without departing from the scope of the invention. Similarly, other liquids may be used besides water without departing from the scope of the invention.

The operations of system 970 also include apportioning the slurry 974 and forming a cleaning slug. In one specific example, the control sub-system 917 (e.g., computing device) uses pump 990 to provide the cleaning slug to the delivery chamber 983. In other embodiments (e.g., if pump 990 is omitted), the control sub-system 917 closes the slurry valve 992 and opens at least one of the control valves 915. The control sub-system 917 then commands the system 970 to create a positive pressure difference between the holding chamber 972 and the delivery chamber 983 and then open the slurry valve 992. The created positive pressure difference pushes a flow of slurry, already created in the holding chamber 972, into the delivery chamber 983. The slurry valve 992 then closes and stops the flow of slurry from holding chamber 972 into the delivery chamber 983 and a cleaning slug of slurry is therefore defined.

It can be appreciated that creating a pressure difference between the holding chamber 972 and the delivery chamber 983 can be done in variety of ways. For example, the positive pressure difference can be achieved by controlling the pressure of gas or liquid using pumps, pressure regulators, proportional pressure regulators (PPR) or electrical pressure regulators (EPR). For example, in one specific and illustrative arrangement, the gas pressure can be controlled using two proportional pressure regulators, namely a primary PPR 919, and a secondary PPR 921. The primary PPR 919 is positioned between the primary gas supply 985A and the primary gas port 903 and controls the gas pressure in the delivery chamber 983. The secondary PPR 921 is positioned between the secondary gas supply 985B and secondary gas port 907 and controls the gas pressure of the holding chamber 972. The PPRs 919 and 921 are controlled by the automated control to create a selectable positive pressure difference between the holding chamber and the delivery chamber.

As noted, the system 970 delivers the cleaning slug (apportioned amount of the liquid-powder mixture) through at least a portion of a channel of the endoscope 100 to be cleaned. Delivery of a cleaning slug into the endoscope channel can occur in a variety of ways. For example, before the apportioned liquid-powder mixture is transferred into the delivery chamber 983, carrier fluid from primary gas supply 984B is supplied to the delivery chamber 983 at a regulated pressure using primary PPR 919. The carrier fluid (e.g., gas) may propel the apportioned liquid-powder mixture into a selected internal channel of endoscope 100 through the distribution manifold 994, which has already selected one of the control valves 915 to be opened at this time (e.g., the cleaning slug is accelerated to the appropriate velocity). Once at the bottom of the delivery chamber 983, the cleaning slug exits the delivery chamber, enters the distribution manifold 994, and then enters a selected internal channel of the endoscope 100 at an appropriate velocity. The process may be repeated many times for an internal channel of the endoscope 100 before moving on to the next internal channel closing one control valve 915 and opening another control valve 915.

Another technique for delivering a cleaning slug of slurry into the target lumen in FIG. 9 uses a self-regulating bi-stable process. In such examples, the carrier fluid from primary gas supply 985B is again supplied to the delivery chamber 983 at a regulated pressure using primary PPR 919. Once the slurry valve 992 opens, a cleaning slug of slurry may transfer into the delivery chamber 983. It can be appreciated that the transfer of the first cleaning slug can happen automatically (e.g., because of gravity) or may be assisted by pump 990 and/or creation of a positive pressure difference between the holding chamber 972 and the delivery chamber 983. If used, the positive pressure difference may naturally be brought about by the high flow of air through the endoscope due to the low fluidic resistance of an un-occluded lumen. Once this cleaning slug occludes the exit of the delivery chamber, the pressure of the delivery chamber 983 will increase due to the increased fluidic resistance of the fluidic path downstream of the delivery chamber. As a result, the holding chamber 972 and delivery chamber 983 will settle at a similar pressure, which in turn, stops any additional flow of slurry into the delivery chamber. The cleaning slug then proceeds through the selected internal channel to then exit the endoscope 100. When the cleaning slug of slurry exits the endoscope channel, the pressure of the delivery chamber 983 will drop because of the lower fluidic resistance of the downstream fluidic line and the limited flow rate of the carrier gas. This will result in a pressure difference between the delivery chamber 983 and the holding chamber 972. As a result, another cleaning slug of slurry will be drawn from the holding chamber 972. The newly drawn cleaning slug is again acted upon by gas flow to propel the slug through the delivery chamber 983, distribution manifold 994, and into a selected internal channel of the endoscope 100.

In general, as long as the flow of pressure-regulated gas continues and the supply of slurry in the holding chamber is not finished, the process will essentially continue automatically with the manifold 994 selecting which internal channel will receive the cleaning slug of slurry. More specifically, once the cleaning slug has cleared through an internal channel, the control sub-system can change the states of control valves 915 and the process is repeated for one or more endoscope channels. In a variation of this, the process may be repeated multiple times through the same internal channel until a sufficient level of debris removal is achieved before moving on to another channel.

By selecting the appropriate parameters such as size of the chambers, amount of liquid, amount of powder, and gas pressure, the system can self-regulate and prevent endoscope internal channel blockage while cleaning the endoscope. In some embodiments, the size of the cleaning slug can more deliberately be altered by changing the gas pressure, amount of water, amount of powder, size of the chambers, etc.

In a variation of the above process according to a further embodiment, the slurry valve 992 and all control valves 915 associated with each connector 923, are initially closed. Similar to the previous technique, once the slurry is formed in the holding chamber 972 using one of the earlier mentioned processes, a positive pressure difference is created between the holding chamber and the delivery chamber using primary and secondary proportional pressure regulators (PPRs) or other means such as electrical pressure regulators (EPRs). Then, the slurry valve 992 opens and the positive pressure difference draws a cleaning slug of slurry into the delivery chamber. The slurry valve then closes and stops the flow of slurry from the holding chamber into the delivery chamber to define a cleaning slug of slurry. A pressure-regulated gas, typically compressed air, is then introduced into the delivery chamber 983 through primary gas port 903 to again create flow of gas. However, unlike the previous method, the cleaning slug is retained in the delivery chamber 983 until one of the control valves 915 in the distribution manifold 994 is opened. After a selectable time, one of the control valves 915 is opened and the cleaning slug is propelled at an appropriate velocity into an endoscope channel using pressurized air. By doing so, the cleaning efficiency may be enhanced by providing a better control over the size of the cleaning slug because the cleaning slug will be fully defined within the delivery chamber before it will be propelled as whole rather than being partially propelled. The described high velocity cleaning slugs of slurry can use, for example, their composition and velocity created by the pressurized gas, water or mixture of gas and water in the delivery chamber, to create a strong physical cleaning action across the internal walls of the selected endoscope channel.

It should be appreciated that the systems and methods of cleaning can provide a means to clean the internal channels of a medical device efficiently. The degree of contamination within the medical device after cleaning can be such that it meets all relevant standards and can be substantially better than using prior art means. Once the cleaning process is completed, the internal channels may be rinsed by flowing water and/or gas through each internal channel in a similar way to the flushing process described above. In certain embodiments, the cleaning process may be substantially automatic after initial setup, and its operation can be very simple for an operator. Advantageously, using computer control of all the valves, ports, and pumps, the cleaning time can be optimized in order to minimize the down time of the medical device.

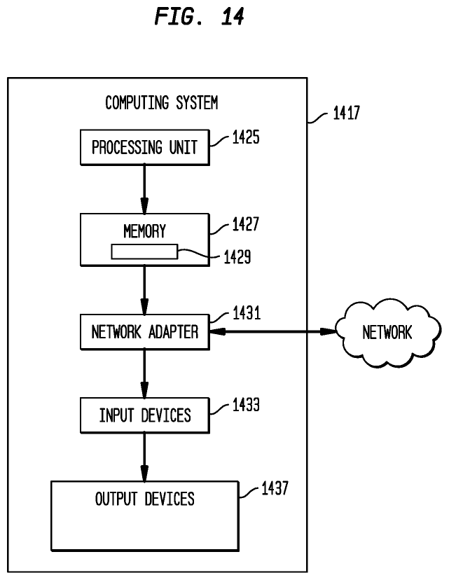

As noted above, a lumen cleaning system in accordance with embodiments presented herein can include, or be controlled by, a control sub-system. FIG. 14 is block diagram illustrating an example computing device 1417 configured to operate as a control sub-system for a lumen cleaning system, in accordance with certain embodiments presented herein. The computing device 1417 can comprise, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 1417 can be a single virtual or physical device operating in a networked environment over communication links to one or more remote devices, such as an implantable medical device or implantable medical device system.

In its most basic configuration, computing device 1417 includes at least one processing unit 1425 and memory 1427. The processing unit 1425 includes one or more hardware or software processors (e.g., Central Processing Units) that can obtain and execute instructions. The processing unit 1425 can communicate with and control the performance of other components of the computing system 1417.

The memory 1427 is one or more software or hardware-based computer-readable storage media operable to store information accessible by the processing unit 1425. The memory 1427 can store, among other things, instructions executable by the processing unit 1425 to implement applications or cause performance of operations described herein, as well as other data. The memory 1427 can be volatile memory (e.g., RAM), non-volatile memory (e.g., ROM), or combinations thereof. The memory 1427 can include transitory memory or non-transitory memory. The memory 1427 can also include one or more removable or non-removable storage devices. In examples, the memory 1427 can include RAM, ROM, EEPROM (Electronically-Erasable Programmable Read-Only Memory), flash memory, optical disc storage, magnetic storage, solid state storage, or any other memory media usable to store information for later access. In examples, the memory 1427 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 1427 can include wired media such as a wired network or direct-wired connection, and wireless media such as acoustic, RF, infrared and other wireless media or combinations thereof. In certain embodiments, the memory 1427 comprises lumen cleaning control logic 1429 that, when executed, enables the processing unit 1425 to perform aspects of the techniques presented.

In the illustrated example, the system 1417 further includes a network adapter 1431, one or more input devices 1433, and one or more output devices 1435. The system 1417 can include other components, such as a system bus, component interfaces, a graphics system, a power source (e.g., a battery), among other components. The network adapter 1431 is a component of the computing system 1417 that provides network access (e.g., access to at least one network). The network adapter 1431 can provide wired or wireless network access and can support one or more of a variety of communication technologies and protocols, such as ETHERNET, cellular, BLUETOOTH, near-field communication, and RF (Radiofrequency), among others. The network adapter 1431 can include one or more antennas and associated components configured for wireless communication according to one or more wireless communication technologies and protocols.

The one or more input devices 1433 are devices over which the computing system 1417 receives input from a user. The one or more input devices 1433 can include physically-actuatable user-interface elements (e.g., buttons, switches, or dials), touch screens, keyboards, mice, pens, and voice input devices, among others input devices. The one or more output devices 1435 are devices by which the computing system 1417 is able to provide output to a user. The output devices 1435 can include, a display, one or more speakers, among other output devices.

It is to be appreciated that the arrangement for computing system 1417 shown in FIG. 14 is merely illustrative and that aspects of the techniques presented herein may be implemented at a number of different types of systems/devices. For example, the computing system 1417 could be a laptop computer, tablet computer, mobile phone, surgical system, etc.

As noted, aspects of the techniques presented herein are used to deliver so-called ‘cleaning slugs’ (e.g., apportioned amounts of a liquid-powder mixture) to a target lumen in order to clear/clean the lumen of contaminants (e.g., bioburdens). The size, fluidity, velocity, and/or other characteristics/attributes of the cleaning slugs are determined such that, as the cleaning slugs travel through the target lumen (i.e., the lumen to be cleaned), the cleaning slugs will interact with (e.g., scour) the walls of the target lumen to remove the contaminants disposed on the inner surface/walls of the lumen

In certain arrangements, the cleaning slug attributes are determined based on the fluidic resistance of at least a proximal portion of the target lumen to be cleaned. The fluidic resistance is the tendency for a fluidic pathway to resist flow of a given fluid due to the combined geometric and surface properties of the pathway. Examples of these properties include the dimensions of the target lumen to be cleaned, where the dimensions include one or both of the cross-sectional width (e.g., diameter) of the target lumen and the length of the target lumen, the internal surface roughness of the lumen, etc. As noted, several different methods of calculating the fluidic resistance exist and are described in greater detail in Australian Patent Application No. 2021901734, entitled “Systems and Methods for the Identification, Evaluation, and/or Closed-Loop Cleaning of lumens,” filed Jun. 9, 2021, and concurrently filed patent application entitled “Systems and Methods for the Identification, Evaluation, and/or Closed-Loop Reprocessing of lumens,” filed Jun. 9, 2022. The content of both of these applications is hereby incorporated by reference herein.

Certain lumens have a substantially constant internal dimension (cross-sectional width) along the elongate length thereof. With constant width lumens, the cleaning slug parameters determined at the time of delivery are generally sufficient to ensure sufficient cleaning of the entire length of the lumen (i.e., from the proximal end to the distal end of the lumen). For example, with a constant width lumen, the fluidity and size of the lumen can remain substantially constant as the cleaning slug travels through the lumen. Moreover, since the constant width of the lumen and the length of the lumen is known, the cleaning slug can be delivered with a suitable velocity so that the cleaning slug will reach the distal end in an acceptable period of time and such that the velocity is optimal to remove contaminants from the walls of the lumen as the cleaning slug travels through the lumen.

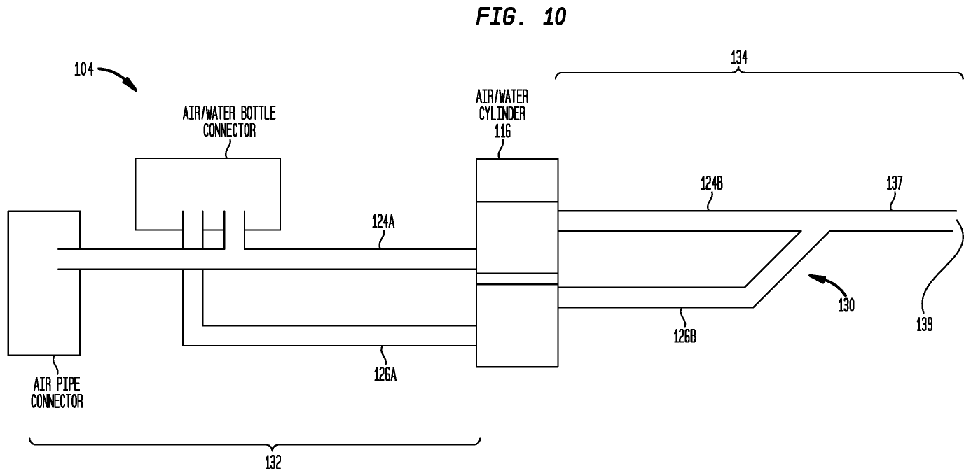

However, not all medical device lumens have constant internal dimensions the length of the lumen. Instead, certain medical device lumens have variable internal dimensions, certain lumens merge together within a device, etc., which create complex fluidic paths for cleaning slugs. For example, FIG. 10 is a schematic diagram illustrating specific examples of complex fluidic paths resulting from variable internal dimensions of lumens with reference to endoscope 100 of FIG. 1 and, more specifically, with reference to the air channel 124 and the water channel 126 of the endoscope 100. The air channel 124 and the water channel 126 are sometimes collectively referred to as the “air/water channel” of the endoscope 100. However, for ease of description, the air channel 124 and the water channel 126 will be described and referred to herein separately.

It is to be appreciated that specific reference to the air and water channels of an endoscope (e.g., air channel 124 and the water channel 126) in FIG. 10, as well as other embodiments presented herein, is merely illustrative and the invention is not limited to use with these specific lumens or to only use with endoscopes in general. As such, it is to be appreciated that the techniques presented herein can be used to clean different complex lumens of different devices/instruments used in any of a number of different applications.

Shown in FIG. 10 is a schematic representation of the air channel 124 and the water channel 126. As noted above, the air channel 124 includes two sections, referred to as proximal section 124A and distal section 124B that are connected via the air/water valve 116, while the water channel 126 similarly includes two sections, referred to as proximal section 126A and distal section 126B that are also connected via the air/water valve 116. The distal section 126B of the water channel joins to the distal section 124B of the air channel at a location 130 within the distal end 102 of the endoscope 100.

As shown, the proximal section 124A of the air channel 124 and the proximal section 126A of the water channel 126 each extend from the connector end 104 of the endoscope 100 (e.g., the air/water bottle connector and the air pipe connector) to the air/water valve 116. The distal section 124B of the air channel 124, as well as the distal section 126B of the water channel 126 each extend from the air/water valve 116 to the location 130 within the distal end 102 of the endoscope 100. Location 130 is the location/point at which the distal section 124B of the air channel 124 and the distal section 126B of the water channel merge to form a merged exit channel 137. As noted, the proximal sections 124A and 126A of the channels 124 and 126 are sometimes referred to as being located within a universal cord section (cord) 132 of the endoscope 100, while the distal sections 124B and 126B of the channels 124 and 126 are sometimes referred to as being located within an insertion tube 134 of the endoscope. For ease of illustration, the biopsy/suction channel 122 and the water-jet channel 128 have been omitted from FIG. 10.

Also shown in FIG. 10 are example dimensions, including internal dimensions and lengths, for each section of the air channel 124 and the water channel 126. It is to be appreciated that the example dimensions shown in FIG. 10 are merely illustrative and that the techniques presented herein can be used with a variety of other lumens having different dimensions.

In the specific example of FIG. 10, the proximal section 124A of the air channel 124 has an internal dimension (ID) (e.g., internal diameter) of approximately 2.0 millimeters (mm) and length of approximately 1.5 meters (m), while the distal section 124B of the air channel 124 has an internal dimension of approximately 1.4 mm and length of approximately 1.5 m. In addition, the proximal section 126A of the water channel 126 has an internal dimension (e.g., internal diameter) of approximately 2.4 mm and length of approximately 1.5 m, while the distal section 126B of the water channel 126 has an internal dimension of approximately 1.4 mm and length of approximately 1.5 m. The merged exit channel 137 has an internal dimension of approximately 1.0 mm, and a length of approximately 0.185 m.

In other words, the proximal sections 124A/126A of the air channel 124 and the water channel 126 have internal dimensions that are larger than the internal dimension of the corresponding distal sections 124B/126B (e.g., after the air/water cylinder 116, the lumens narrow down). The variable internal dimensions of each of the air channel 124 and the water channel 126 creates problems for cleaning these lumens with cleaning slugs delivered from the connector end 104 of the endoscope 100. As noted above, there can be multiple connectors, such as one connector for the air pipe and one connector for the air/water bottle. In particular, cleaning slugs having attributes suitable for cleaning the larger proximal sections 124A and 126A of the air channel 124 and water channel 126, respectively, may clog the narrower corresponding distal sections 124B and 126B (e.g., cleaning slugs configured for the 2.0 mm and 2.4 mm ID sections of the lumens will highly likely clog the 1.4 mm and 1.0 mm ID sections of the lumens). However, cleaning slugs that are configured such that they won't clog the distal sections 124B and 126B of the air channel 124 and water channel 126, respectively, may not able to effectively clean the proximal sections 124A and 126A of the air channel 124 and water channel 126, respectively (e.g., cleaning slugs configured for the 1.4 mm and 1.0 mm ID sections of the lumens will pass through the 2.0 mm and 2.4 mm ID sections without sufficiently interacting with the walls to provide the needed action). In addition, the merging of the distal sections 124A and 126B, as well as the small nozzle 139 at the distal end adds to the fluidic complexity.

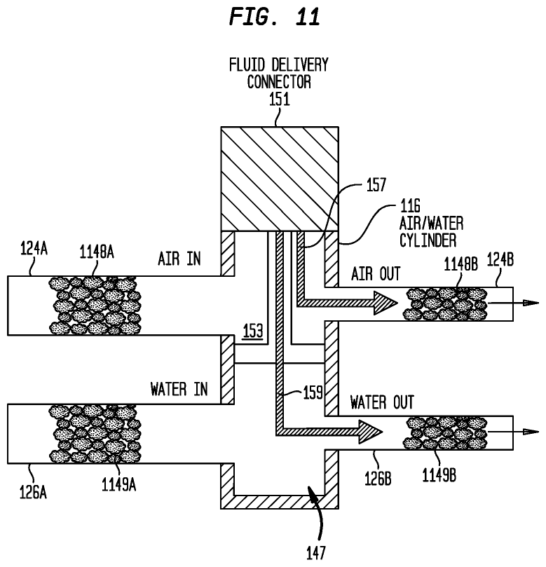

Presented herein are techniques for cleaning fluidically complex lumens (e.g., lumens channels having, for example, variable internal dimensions) via cleaning slugs delivered from a proximal end of the lumen. These techniques are described in greater detail with reference to FIGS. 11-15. For ease of description, the examples of FIGS. 11-15 will be described with reference to the air channel 124 and water channel 126 of endoscope 100.

Referring first to FIG. 11, shown is an arrangement for cleaning fluidically complex lumens via modulation of one or more attributes of the cleaning slugs during the cleaning process (e.g., modulation of the cleaning slug attributes at one or more locations between a proximal end and a distal end of a lumen). More specifically, shown in FIG. 11 is the air/water cylinder 116, which defines an interior volume/opening 147, as well as portions of each of the air channel 124 and water channel 126 connected to the air/water cylinder 116 (e.g., portions of proximal section 124A of the air channel 124, proximal section 126A of the water channel 126, distal section 124B of the air channel 124, and distal section 126B of the water channel 126).

Also shown in FIG. 11 is a fluid delivery connector 151 that is configured to mechanically mate with (attach to) the air/water valve 116. As shown, the fluid delivery connector 151 is configured to bifurcate (separate) the interior volume 147 of the air/water cylinder 116 into two chambers, referred to herein as air chamber (or first chamber) 153 and water chamber (or second chamber) 155. For example, as shown in FIG. 11, the fluid delivery connector 151 includes a separator portion 157 that fluidically isolates the air chamber 153 from the water chamber 155. The separator portion 157 can, for example, allow accurate blockage detection of individual channels. Further aspects of exemplary fluid delivery connectors that can be used with embodiments of the present invention are described in greater detail in International Patent Application No. PCT/AU2022/050547 entitled “Medical Device Port Connectors,” filed on Jun. 3, 2022, the content of which is incorporated reference herein.

In addition to bifurcating the interior volume 147 of the air/water cylinder 116, the fluid delivery connector 151 is also configured to separately deliver a fluid (e.g., water) to each of the air chamber 153 from the water chamber 155. This fluid delivery from a fluid source (not shown in FIG. 11) is schematically represented in FIG. 11 by arrows 157 and 159.

As shown in FIG. 11, a cleaning slug 1148A is delivered to the connector end of the proximal section 124A of the air channel 124. The cleaning slug 1148A is configured (e.g., sized) such that the cleaning slug 1148A will physically interact with the walls of the proximal section 124A as it passes there through. After a period of time, the cleaning slug 1148A will reach the air/water cylinder 116 and, more specifically, the air chamber 153 formed by the fluid delivery connector 151. As noted above, and as schematically shown in FIG. 11, the proximal section 124A of the air channel 124 is relatively larger than the distal section 124B of the air channel. As a result, the cleaning slug 1148A, if permitted to continue into the distal section 124B of the air channel 124, may clog the air channel (e.g., get stuck within the distal section 124B).

To address this issue, the techniques presented herein modulate the attributes of the cleaning slug 1148A within the air chamber 153 to form a modulated/modified cleaning slug 1148B that is specifically configured (e.g., sized) for cleaning the smaller distal section 124B. In particular, in this example, fluid 157 is added to the air chamber 153 to, for example, modulate/change the fluidity (e.g., dilute) the cleaning slug 1148A to form the modulated cleaning slug 1148B, which is smaller than the cleaning slug 1148A and, as such, is appropriately sized for cleaning the distal section 124B of the air channel 124. More generally, these operations change the ratio of the powder to the liquid, where the higher liquid to powder ratio enables the cleaning slug 1148B to flow through the narrower distal section 124B more readily.

A similar approach is applied with the water channel 126 where a cleaning slug 1149A is delivered to the connector end of the proximal section 126A of the water channel. The cleaning slug 1149A is configured (e.g., sized) such that the cleaning slug 1149A will physically interact with the walls of the proximal section 126A as it passes there through. After a period of time, the cleaning slug 1149A will reach the air/water cylinder 116 and, more specifically, the water chamber 155 formed by the fluid delivery connector 151. As noted above, and as schematically shown in FIG. 11, the proximal section 126A of the water channel 126 is relatively larger than the distal section 126B of the water channel. As a result, the cleaning slug 1149A, if permitted to continue into the distal section 126B of the water channel 126, is likely to clog the water channel (e.g., get stuck within the distal section 126B).

As noted above, to address this issue, the techniques presented herein modulate the attributes of the cleaning slug 1149A within the water chamber 155 to form a modulated/modified cleaning slug 1149B that is specifically configured (e.g., sized) for cleaning the smaller distal section 126B. In particular, in this example, fluid 159 is added to the water chamber 155 to, for example, dilute the cleaning slug 1149A to form the modulated cleaning slug 1149B, which is smaller than the cleaning slug 1149A and, as such, is appropriately sized for cleaning the distal section 126B of the water channel 126.

As noted, FIG. 11 generally illustrates techniques for modulating (e.g., diluting) relatively larger cleaning slugs before pushing them into smaller sections of the endoscope 100. The relatively larger cleaning slugs can remove contaminants from the proximal sections of the air and water channels (air-in and water-in sections) before being modulated within the bifurcated air/water cylinder into relatively smaller cleaning slugs that can remove contaminants from the distal sections of the air and water channel, without clogging the relatively smaller distal section. In general, the modulation applied to the cleaning slug can be determined based on the characteristics of the proximal section relative to distal section of the lumen, such as the relative internal dimension differences between the proximal section and distal section of the lumen (e.g., based on the fluidic resistance of the proximal section relative to the fluidic resistance of the distal section). For example, a relatively larger internal dimension difference between a proximal section and a distal section may require relatively greater modulation (e.g., dilution) of the cleaning slug.

FIG. 11 has been described with reference to the air channel 124 and water channel 126 of endoscope 100, where the air channel 124 and water channel 126 each comprise proximal and distal sections fluidically connected via the air/water cylinder 116. As noted, it is to be appreciated that this use is merely illustrative and that the embodiments of FIG. 11 can be used with any of a number of different lumens.

Moreover, it is to be appreciated that the techniques of FIG. 11 can be used with any combination of fluidically connected lumens, and not necessarily only a lumen having proximal and distal sections. For example, the techniques of FIG. 11 can be used with any combination of a first lumen (or first lumen section) that is fluidically connected to a second lumen (or second lumen section), and where there is, for example, a change in internal dimension between the first lumen and second lumen. In addition, the first lumen (or first lumen section) and second lumen (or second lumen section) can be fluidically connected directly in series (directly connected end-to-end) or indirectly connected in series via an intermediary component, such as a fluidic chamber (e.g., an air/valve). To this end, the term “lumen” is to be broadly understood as any fluidic pathway including at least one fluidic entry point and one or more fluidic exit points. Applying the techniques of FIG. 11, a cleaning slug that is delivered to, and that passes through the first lumen, can be modulated into a modified cleaning slug having, for example, a different size or fluidity, as it transitions into the second lumen.

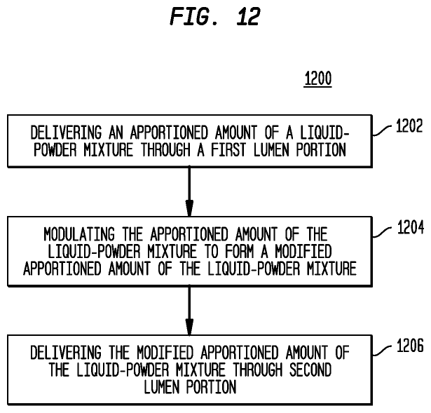

FIG. 12 is a flowchart of an example method 1200 for cleaning a lumen in accordance with embodiments of the present invention. Method 1200 begins at 1202 where an apportioned amount of a liquid-powder mixture (i.e., cleaning slug) is delivered through a first lumen portion, which can comprise a first lumen or a proximal section of a first lumen. At 1204, the apportioned amount of the liquid-powder mixture is modulated (e.g., diluted) to form a modified apportioned amount of the liquid-powder mixture (e.g., modified cleaning slug). At 1206, the modified apportioned amount of the liquid-powder mixture is delivered through a second lumen portion that is fluidically connected to the first lumen portion. The second lumen portion can comprise a second lumen or a distal section of the first lumen.

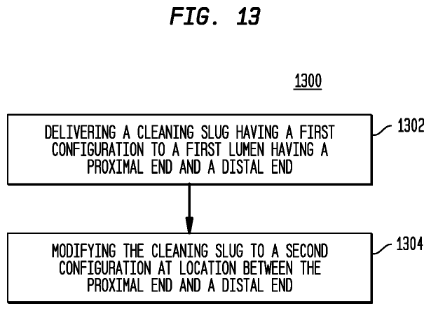

FIG. 13 is a flowchart of an example method 1300 for cleaning a lumen in accordance with embodiments of the invention. Method 1300 begins at 1302 where a cleaning slug having a first configuration is delivered to a first lumen having a proximal end and a distal end and a change in an internal dimension between the proximal end and a distal end. As described elsewhere herein, the “first lumen” need not be a single lumen, but instead can comprise two lumen sections that are fluidically connected to one another. At 1304, the cleaning slug is modified (e.g., diluted) to a second configuration at a location between the proximal end and the distal end of the lumen.

In certain embodiments, the fluidic complexity of channels having, for example, variable internal dimensions can be addressed by increasing the pressure used to propel a cleaning slug through a lumen. In particular, with an increased pressure, the cleaning slug can be “forced” to conform to the size of a narrower lumen. In addition, the use of increased pressure can ensure that the cleaning slugs do not become clogged in the narrower lumens.

Certain aspects of the techniques presented herein have been described with reference to various descriptions of fluid dynamics. It is to be appreciated that these various descriptions are provided for purposes of illustration and that the innovation presented herein works regardless of the believed understanding of the fluid dynamics.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A method for cleaning at least one interior lumen of a medical device comprising:
mixing a liquid with a powder to form a slurry; and
applying at least one flow of fluid to a portion of the slurry to propel the portion of the slurry through the at least one interior lumen of the medical device.

2. The method of claim 1, further comprising:
apportioning the liquid-powder mixture into the portion based at least on a fluidic resistance of at least a proximal section of the at least one lumen.

3. The method of claim 1, wherein mixing the liquid with the powder to form the slurry comprises:
introducing the liquid into a consumable chamber holding the powder.

4. The method of claim 1, wherein applying at least one flow of fluid to a portion of the slurry comprises:
providing the portion of the slurry to a delivery chamber; and
applying the at least one flow of fluid in the delivery chamber to accelerate the portion of the slurry prior to delivery of the portion of the slurry to the at least one interior lumen.

5. The method of claim 4, wherein applying the at least one flow of fluid in the delivery chamber comprises:
applying a first flow of fluid in the delivery chamber and a second flow of fluid in the delivery chamber.

6. The method of claim 1 further comprising:
delivering a fluid flow through the at least one interior lumen without any portion of the slurry.

7. The method of claim 1, further comprising:
mixing the powder with the liquid in at least one holding chamber, wherein the at least one holding chamber is in fluid communication with one or more delivery chambers.

8. The method of claim 7, further comprising:
drawing the portion of the slurry from the at least one holding chamber to at least one of the one or more delivery chambers.

9. The method of claim 7, further comprising:
pumping the portion of the slurry from the at least one holding chamber to at least one of the one or more delivery chambers.

10. (canceled)

11. The method of claim 1, wherein mixing the powder with the liquid to form the slurry comprises:
providing excessive powder relative to the liquid such that undissolved powder is suspended in the slurry.

12. The method of claim 1, wherein mixing the powder with the liquid to form the slurry comprises:
mixing sodium bicarbonate with water.

13. (canceled)

14. (canceled)

15. The method of claim 1, wherein the at least one interior lumen is a fluidically complex lumen having at least a first section with a first internal dimension and a second section fluidically connected with the first section, and wherein the second section has a second internal dimension that is smaller than the first internal dimension.

16. The method of claim 15, further comprising:
modulating at least one attribute of the portion of the slurry at a transition from the first section of the at least one interior lumen to the second section of the at least one interior lumen.

17. The method of claim 16, wherein the second section is connected to the first section via a fluidic chamber, and wherein the method comprises:
modulating the at least one attribute of the portion of the slurry at the fluidic chamber.

18. The method of claim 16, wherein modulating the at least one attribute of the portion of the slurry comprises:
increasing a ratio of fluid to powder of the portion of the slurry.

19. (canceled)

20. (canceled)

21. The method of claim 4, wherein the delivery chamber comprises a frustoconical shaped interior surface, and wherein applying the at least one flow of fluid in the delivery chamber to accelerate the portion of the slurry comprises:
applying the at least one flow of fluid in the at least one delivery chamber to cause the portion to rotate along the frustoconical shaped interior surface.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The method of claim 15, wherein the portion is delivered to the first section with a first configuration, and wherein the method comprises:
modifying the portion to a second configuration at a transition from the first section of the at least one lumen to the second section of the at least one lumen.

34. A system, comprising:
at least one delivery chamber fluidically connected to at least one interior lumen of an apparatus;
at least one of a valve or pump configured to provide an apportioned amount of a liquid-powder mixture to the at least one delivery chamber; and
a delivery mechanism configured to apply at least one flow of fluid to the apportioned amount of the liquid-powder mixture in the at least one delivery chamber to propel the apportioned amount of the liquid-powder mixture through the at least one interior lumen.

35. The system of claim 34, further comprising a control sub-system configured to determine the apportioned amount of the liquid-powder mixture based on a fluidic resistance of at least a proximal section of the at least one interior lumen.

36. The system of claim 34, wherein the at least one delivery chamber comprises a frustoconical shaped interior surface, and wherein the delivery mechanism is configured to apply the at least one flow of fluid in the at least one delivery chamber so that the apportioned amount of the liquid-powder mixture spins along the frustoconical shaped interior surface.

37. The system of claim 34, wherein the at least one delivery mechanism is configured to apply a first flow of fluid in the at least one delivery chamber and a second flow of fluid in the delivery chamber.

38. The system of claim 34, further comprising a holding chamber, wherein the holding chamber is a consumable component that is configured to be mechanically decoupled from the system.

39. The system of claim 34, further comprising a holding chamber, wherein the holding chamber is configured to retain a powder, and wherein the system is configured to deliver a fluid to the holding chamber for mixing with the powder to form the liquid-powder mixture.

40. The system of claim 39, further comprising a motor for use in mixing the fluid with the powder to form the liquid-powder mixture.

41. The system of claim 34, wherein the system is configured to create a pressure difference between a holding chamber and the at least one delivery chamber to draw the apportioned amount of the liquid-powder mixture from the holding chamber to the at least one delivery chamber.

42. The system of claim 34, further comprising:
a distribution manifold fluidly connected between the at least one delivery chamber and the at least one interior lumen.

43. The system of claim 34, wherein the delivery mechanism is configured to apply a flow of at least one of compressed air or water to the apportioned amount of the liquid-powder mixture in the at least one delivery chamber.

44. (canceled)

45. The system of claim 34, wherein the at least one interior lumen is a fluidically complex lumen having at least a first section with a first internal dimension and a second section fluidically connected in series with the first section, and wherein the second section has a second internal dimension that is smaller than the first internal dimension, and wherein the system comprises a fluid delivery connector configured to deliver a fluid at a transition of the at least one interior lumen from the first internal dimension to the second internal dimension to modulate at least one attribute of the apportioned amount of the liquid-powder mixture at the transition of the at least one interior lumen from the first internal dimension to the second internal dimension.

46. (canceled)

47. The system of claim 45, wherein the second section is connected to the first section via a fluidic chamber, and wherein the fluid delivery connector is configured to fluidically connect to the fluidic chamber.

48. The system of claim 47, wherein the fluid delivery connector is configured to bifurcate the fluidic chamber into a first chamber and a second chamber, and wherein the fluid delivery connector is configured to separately deliver the fluid to the first chamber and the second chamber.

49. The system of claim 34, wherein the at least one delivery chamber comprises first and second delivery chambers each separately fluidically connected to a holding chamber.
